(19) United States
(12) Patent Application Publication
(10) Pub. No.: US 2012/0178666 A1
(43) Pub. Date: Jul. 12, 2012

(54) PRODRUGS OF GUANFACINE

(75) Inventors: Richard Franklin, Fleet (GB); Robert G. Tyson, Durham (GB); Bernard T. Golding, Shiremoor (GB); Rhys Whomsley, Basingstoke (GB)

(73) Assignee: SHIRE LLC, Florence, KY (US)

(21) Appl. No.: 13/496,316

(22) PCT Filed: Sep. 15, 2010
(86) PCT No.: PCT/GB2010/051544

§ 371 Date: Mar. 15, 2012

Related U.S. Application Data
(60) Provisional application No. 61/242,507, filed on Sep. 15, 2009.

(30) Foreign Application Priority Data
Sep. 15, 2009 (GB) 0916163.9

(51) Int. Cl. A61K 38/05; C07D 233/88; C07D 207/277; C07D 239/22; A61K 31/197; A61K 31/4172; A61K 31/513; A61P 25/00; A61P 9/00; A61P 9/12; A61P 29/00; A61P 25/18; A61P 25/22; A61P 25/30; A61P 1/00; C07D 207/16; C07C 271/66; A61K 31/401; A61K 31/4015; A61K 31/27; A61K 31/166; C07C 279/22

(52) U.S. Cl. 514/1.3; 562/439; 548/321.5; 548/537; 544/321; 514/563; 514/386; 514/272; 548/532; 560/30; 564/153; 564/157; 514/423; 514/482; 514/616

(57) ABSTRACT

Prodrugs of guanfacine with amino acids or short peptides, pharmaceutical compositions containing such prodrugs and a method for providing therapeutic benefit in the treatment of ADHD/ODD (attention deficient hyperactivity disorder and oppositional defiance disorder) with guanfacine prodrugs are provided herein. Additionally, methods for minimizing or avoiding the adverse gastrointestinal side effects associated with guanfacine administration, as well as improving the pharmacokinetics of guanfacine are provided herein.

### CROSS-REFERENCE TO RELATED APPLICATION

The application claims the benefit of priority from U.S. Provisional Patent Application No. 61/242,507 filed Sep. 15, 2009, the contents of which are incorporated herein by reference.

### FIELD OF THE INVENTION

The present invention relates to various prodrugs of guanfacine. In particular, the present invention relates to amino acid and peptide prodrugs of guanfacine which offer improved pharmacokinetic properties relative to guanfacine itself. The invention also relates to methods of reducing gastrointestinal (GI) side-effects associated with guanfacine therapy. These combined advantages should improve patient compliance and hence the drug's therapeutic effectiveness and patient benefit.

### BACKGROUND OF THE INVENTION

Attention Deficit Hyperactivity Disorder (ADHD) is one of the most common psychiatric conditions affecting children. Prevalence estimates vary but according to data from the National Survey of Children's Health, ˜8% of US children were diagnosed with ADHD in 2003, 56% of whom were treated with medication (Centers for Disease Control and Prevention (2005), Morb. Mortal. Wkly. Rep. 54, 842-847). Psychostimulant medications are the mainstay of therapy for patients with ADHD (Pediatrics (2001), 108, 1033-1044; Arch Gen Psychiatry (1999), 56, 1073-1085; Pediatrics (2004), 113, 754-761). Although >80% of these patients receive stimulant drugs, <40% are reported to exhibit normal behavior with treatment. Additionally, ˜30% of patients either do not respond or cannot tolerate long term therapy with these agents. An additional concern is that these stimulants are classified by the US Drug Enforcement Administration as Schedule II Controlled Substances.

Several classes of non-stimulant drugs appear to be efficacious in patients with ADHD including tricylic antidepressants (imipramine and desipramine), bupropion, a norepinephrine and dopamine reuptake inhibitor, atomoxetine, a norepinephrine re-uptake inhibitor and α-2 adrenoceptor agonists clonidine and guanfacine. The latter has been reported to enhance frontal cortex functioning (PCF) in rats, monkeys and humans. In patients treated for ADHD with guanfacine, the drug may ameliorate prefrontal cortical deficits. Specifically, guanfacine appears to act primarily on the α-2 adrenoceptors in the prefrontal cortex, enhancing working memory, cognitive function and attentiveness.

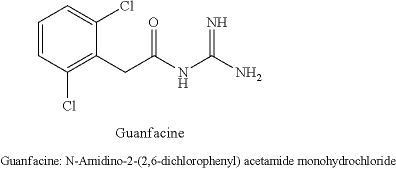

Historically, guanfacine was employed as an antihypertensive agent (TENEX®) due to its effectiveness in lowering blood pressure. Typically, doses of 1-2 mg and occasionally 3 mg/day have been used in the treatment of hypertension. Peak plasma drug levels are reached as early as 1 hour after dosing and may be associated with cardiovascular side effects or somnolence. The drug is usually taken at night to minimize the impact of this. Recently a new guanfacine product (INTUNIV®) has been developed for the treatment of ADHD. This is a sustained release formulation designed to minimize any acute cardiovascular or CNS depressant effects of the drug resulting from the normally rapid rise in plasma drug concentrations. In a recent pharmacokinetic study on INTUNIV® reported by Swearingen et al. (2007), Clin. Therap. 29, 617-624, peak plasma levels were not seen until 6 hours post dosing so minimizing any unwanted cardiovascular or CNS effects.

In common with other α-2 adrenoceptor agonists such as clonidine, guanfacine may inhibit gut motility, leading, in some cases and especially after the higher doses, to constipation. For example, the incidence of constipation reported for the 3 mg dose of TENEX® is ˜15% (FDA label). This may be due in part to a direct local interaction between the drug and α-2 adrenoceptors within the gut. Published data provides evidence not only for the presence of α-2 adrenoceptors in the GI tract and their role in influencing gut motility (Blandizzi (2007), Neurochemistry International, 51, 282-288), but also for a direct effect of selective α-2 adrenoceptor agonists such as UK14,304 on the motility reflexes of guinea pig ileum (Stebbing et al (2001), J of Physiol. 534 465-478). Such effects are clearly undesirable.

INTUNIV® is a controlled release product and one limitation of such formulations is that they may be subject to a food interaction. The presence of food in the stomach serves to raise the gastric pH and slow gastric emptying. This may lead to some erosion of the enteric coating, designed to break down at higher pH's, and some early drug release as a consequence. Administration of INTUNIV® with a high fat meal has been shown to elevate Cₘₐₓ by 75% and increase AUC by 40% (FDA label). While taking the drug under more appropriate prandial conditions may be desirable, this may not always be possible. Variations in the prandial state may therefore lead to some variability in rate and extent of drug exposure.

In spite of the advantages offered by guanfacine, there continues to be a need to reduce side-effects associated with guanfacine therapy. There remains therefore a real need in the treatment of ADHD as well as hypertension for a guanfacine product which retains all the inherent pharmacological advantages of the drug molecule but overcomes its limitations in inducing adverse GI side-effects. The present invention addresses this need.

### SUMMARY OF THE INVENTION

In one aspect of the present invention, there is provided a guanfacine prodrug of Formula (I), or a pharmaceutically acceptable salt or tautomer thereof:

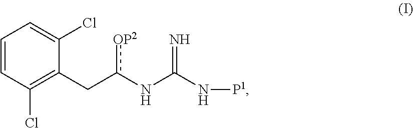

wherein:

P¹ is hydrogen or -L-R;

P² is absent, hydrogen or -L-R;

provided that when P¹ is hydrogen, P² is not absent;

L is absent, or a group selected from the group comprising:

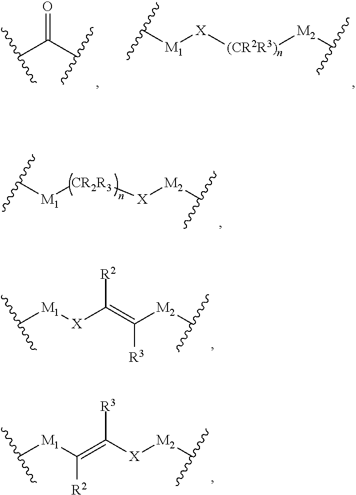

an amino acid residue containing from 2 to 20 carbon atoms, and a peptide formed from 2 to 10 independently selected amino acids each containing from 2 to 20 carbon atoms;
wherein:

M₁ is absent or is selected from the group comprising: —CH₂—,

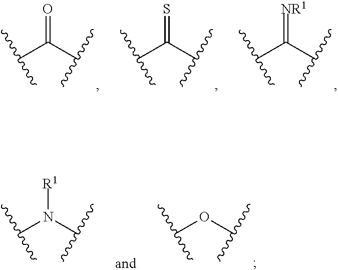

wherein R¹ is selected from the group comprising: H, C₁₋₄ alkyl and C₃₋₈ cycloalkyl;

M₂ is absent or is selected from the group comprising: —CH₂—,

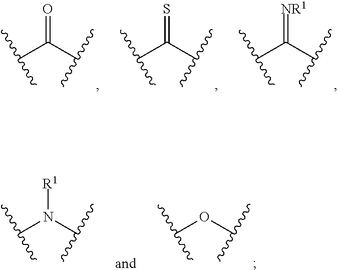

wherein R¹ is selected from the group comprising: H, C₁₋₄ alkyl and C₃₋₈ cycloalkyl;

R² and R³ are each independently selected at each occurrence from the group comprising: hydrogen, hydroxy, C₁₋₆ alkoxy, C₁₋₆ alkyl C₁₋₆ alkoxy, —(CR⁴R⁵)ₙOC(═O)R⁶, —(CR⁴R⁵)ₙC(═O)R⁶, —C(═O)R⁶, C₁₋₆ alkyl, C₁₋₆ haloalkyl, aryl, —N R⁴R⁵ and —NR⁴(CO)R⁶; or together with the atom to which they are bonded, R² and R³ may form a carbonyl, an ethylene or a C₃₋₆ cycloalkyl;

R⁴ and R⁵ are each independently selected from the group comprising: H, C₁₋₆ alkyl, C₁₋₆ haloalkyl, C₃₋₈ cycloalkyl and phenyl;

R⁶ is selected from the group comprising: hydroxyl, C₁₋₆ alkyl, C₁₋₆ alkoxy, C₃₋₈ cycloalkyl and phenyl;

X is selected from the group comprising: a bond, —O—, —NH—, —CR²R³— and a saturated or unsaturated ring having from 3 to 6 carbon atoms in the ring;

R is hydroxy, an amino acid residue containing from 2 to 20 carbon atoms or a peptide formed from 2 to 10 independently selected amino acids each containing from 2 to 20 carbon atoms, or R is a group selected from the group comprising: —NH₂ and —NR⁴R⁵; and

n is at each occurance independently an integer of 0-16.

In an embodiment, there is provided a guanfacine prodrug of Formula (I), or a pharmaceutically acceptable salt or tautomer thereof:

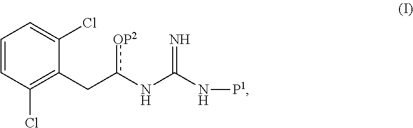

wherein:

P¹ is hydrogen or -L-R;

P² is absent, hydrogen or -L-R;

provided that when P¹ is hydrogen, P² is not absent;

L is absent, or a group selected from the group comprising:

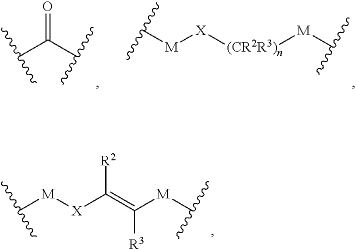

an amino acid residue containing from 2 to 20 carbon atoms, and a peptide formed from 2 to 10 independently selected amino acids each containing from 2 to 20 carbon atoms; wherein:

each M is independently absent or independently selected at each occurrence from the group comprising: —CH₂—,

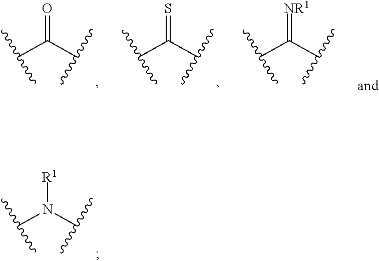

wherein R¹ is selected from the group comprising: H, C₁₋₄ alkyl and C₃₋₈ cycloalkyl;

R² and R³ are each independently selected at each occurrence from the group comprising: hydrogen, hydroxy, C₁₋₆ alkoxy, C₁₋₆ alkyl C₁₋₆ alkoxy, —(CR⁴R⁵)ₙOC(═O)R⁶, —C(═O)R⁶, C₁₋₆ alkyl, C₁₋₆ haloalkyl, aryl, —NR⁴R⁵ and —NR⁴(CO)R⁶; or together with the atom to which they are bonded, R² and R³ may form a C₃₋₆ cycloalkyl;

R⁴ and R⁵ are each independently selected from the group comprising: H, C₁₋₆ alkyl, C₁₋₆ haloalkyl, C₃₋₈ cycloalkyl and phenyl;

R⁶ is selected from the group comprising: hydroxyl, C₁₋₆ alkyl, C₁₋₆ alkoxy, C₃₋₈ cycloalkyl and phenyl;

X is selected from the group comprising: a bond, —O— and —NH—;

R is hydroxy, an amino acid residue containing from 2 to 20 carbon atoms or a peptide formed from 2 to 10 independently selected amino acids each containing from 2 to 20 carbon atoms, or R is a group selected from the group comprising: —NH₂ and —NR⁴R⁵; and

n is at each occurance independently an integer of 0-10.

The combinations of the L and R groups contemplated within the scope of the present invention include those in which combinations of variables (and substituents) of the L and R groups are permissible so that such combinations result in stable compounds of Formula (I). For purposes of the present invention, it is understood that the combinations of the variables can be selected by one of ordinary skill in the art to provide compounds of Formula (I) that are chemically stable and that can be readily synthesized by techniques known in the art, as well as those methods set forth in the example section and figures.

The invention encompasses tautomeric forms of the compounds of Formula (I) as well as geometrical and optical isomers. Thus, it is contemplated that the present invention specifically includes tautomers of Formula (I) or pharmaceutically acceptable salts thereof. For example, the prodrugs described herein can exist in tautomeric form with respect to the carbonyl group and the guanidino group in guanfacine. Additionally, when the compounds of Formula (I) include an alkene double bond (for example, compounds of Formula (I) having L as

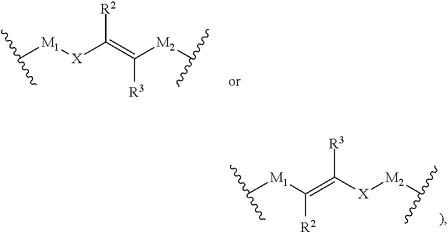

the illustrated structures are intended to include both the E- and Z-geometrical isomers.

In an embodiment, the compound of Formula (I) may have a structure according to Formula (II):

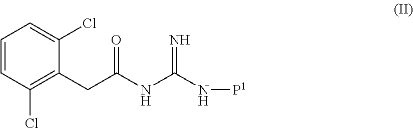

or tautomer thereof,

wherein

P¹ is L-R.

In this regard, the prodrugs have a structure

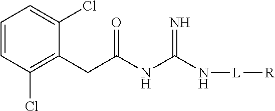

or tautomer thereof.

In an embodiment, n is independently selected at each occurance from the value 0, 1, 2, 3 or 4. In one embodiment, n is 0. In another embodiment, n is 1. In a further embodiment, n is 2. In yet another embodiment, n is 3. In still further embodiment, n is 4.

In an embodiment, L is

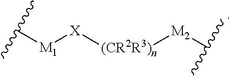

In an embodiment, M₁ is

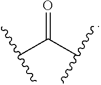

In an embodiment, M₂ is

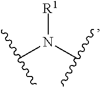

preferably M₂ is

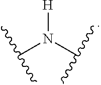

In an embodiment, M₂ is

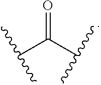

In a preferred embodiment, L is

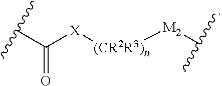

In a particularly preferred embodiment, L is

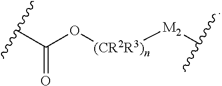

In another particularly preferred embodiment, L is

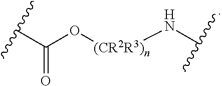

In an embodiment, R² and R³ are each independently selected at each occurance from the group comprising: H, C₁₋₃ alkyl (e.g. methyl, ethyl, i-propyl) and —C(═O)R⁶. Preferably, R⁶ is —OH.

In an embodiment, L is a moiety selected from those recited in the following table:

TABLE 1

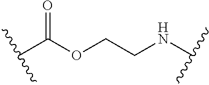

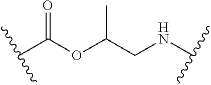

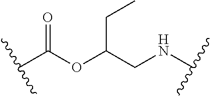

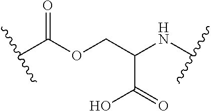

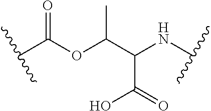

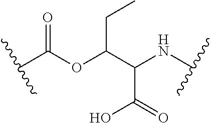

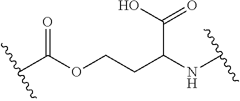

In an embodiment, L is

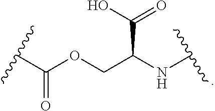

In an embodiment, L is

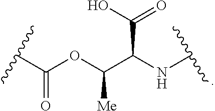

In another embodiment, L is

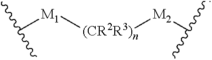

In a preferred embodiment, L is

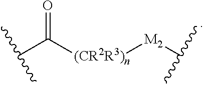

In a particularly preferred embodiment, L is

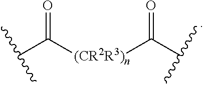

In an embodiment, R² and R³ are each independently selected at each occurance from the group comprising: H, —OH and —C(═O)R⁶. Preferably, R⁶ is —OH.

In an embodiment, L is a residue including a dicarboxylic acid moiety. It is noted that the actual carboxylic acid (i.e. prior to its attachment between guanfacine and R) is recited in the table below:

TABLE 2

| Common Name | IUPAC Name | Chemical Formula |
|---|---|---|
| Oxalic Acid | Ethanedioic Acid | HOOC—COOH |
| Malonic Acid | Propanedioic Acid | HOOC—(CH₂)—COOH |
| Succinic Acid | Butanedioic Acid | HOOC—(CH₂)₂—COOH |
| Glutaric Acid | Pentanedioic Acid | HOOC—(CH₂)₃—COOH |
| Adipic Acid | Hexanedioic Acid | HOOC—(CH₂)₄—COOH |
| Pimelic Acid | Heptanedioic Acid | HOOC—(CH₂)₅—COOH |
| Suberic Acid | Octanedioic Acid | HOOC—(CH₂)₆—COOH |
| Azelaic Acid | Nonanedioic Acid | HOOC—(CH₂)₇—COOH |
| Sebacic Acid | Decanedioic Acid | HOOC—(CH₂)₈—COOH |
| Undecanedioic Acid | Undecanedioic Acid | HOOC—(CH₂)₉—COOH |
| Dodecanedioic Acid | Dodecanedioic Acid | HOOC—(CH₂)₁₀—COOH |
| Brassylic Acid | Tridecanedioic Acid | HOOC—(CH₂)₁₁—COOH |
| | 1,11-Undecanedicarboxylic Acid | |
| Tetradecanedioic Acid | 1,12-Dodecanedicarboxylic Acid | HOOC—(CH₂)₁₂—COOH |
| Pentadecanedioic Acid | 1,15-Pentadecanedioic Acid | HOOC—(CH₂)₁₃—COOH |
| Thapsic Acid | Hexadecanedioic Acid | HOOC—(CH₂)₁₄—COOH |
| | Hexane-1,16-dioic Acid | |
| Heptadecanedioic Acid | 1,15-Pentadecanedicarboxylic Acid | HOOC—(CH₂)₁₅—COOH |
| Octadecanedioic Acid | 1,16-Tetradecanedicarboxylic Acid | HOOC—(CH₂)₁₆—COOH |
| Phthalic Acid | Benzene-1,2-Dicarboxylic Acid | C₆H₄(COOH)₂ |
| Terephthalic Acid | Benzene-1,4-Dicarboxylic Acid | C₆H₄(COOH)₂ |
| Aconitic Acid | Prop-1-ene-1,2,3-tricarboxylic acid | C₆H₆O₆ |
| Achilleic Acid | | |
| Citraconic Acid | 2-methylbut-2-enedioic acid | C₅H₆O₄ |
| Itaconic Acid | Methylenesuccinic Acid | C₅H₆O₄ |
| | 2-Methylidenebutanedioic acid | |
| Aconitic Acid | Prop-1-ene-1,2,3-tricarboxylic acid | C₆H₆O₆ |
| α-Ketoglutaric Acid | 2-oxopentanedioic acid | C₅H₆O₅ |
| N^{α}-Acetyl glutamatic acid | 2-acetamidopentanedioic acid | C₇H₁₁NO₅ |
| Isocitric acid | 1-Hydroxypropane-1,2,3-tricarboxylic acid | C₆H₈O₇ |
| 2-hydroxy-3-methylsuccinic | 2-hydroxy-3-methylsuccinic acid | C₅H₉O₅ |
| acid | | |
| 2-hydroxy-2,3-dimethylsuccinic | 2-hydroxy-2,3-dimethylsuccinic acid | C₆H₁₀O₅ |
| acid | | |
| citric acid | 2-hydroxypropane-1,2,3-tricarboxylic acid | C₆H₈O₇ |

In an embodiment, L is a residue which includes both M¹ and M² as

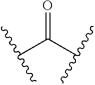

and has a structure as recited in the table below:

TABLE 3

| Name of L residue | Structure |
|---|---|
| N^{a}-Acetyl Aspartic Acid Linker | 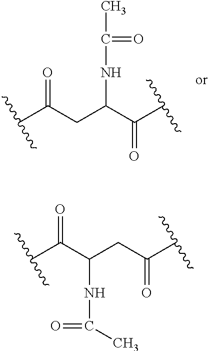 |
| N^{a}-Acetyl Glutamic Acid Linker | 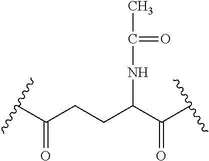 |
| Malic Acid Linker | 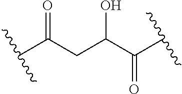 |
| Tartaric Acid Linker | 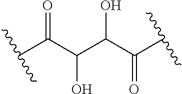 |
| Citramalic Acid Linker | 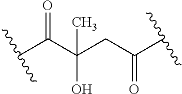 |
| 2-Methyl Succinic Acid Linker | 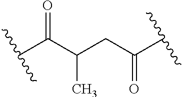 |
| 2,2-Dimethyl Succinic Acid Linker | 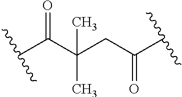 |
| 2,3-Dimethyl Succinic Acid Linker | 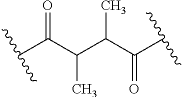 |
| (S)-Citramalic Acid Linker | 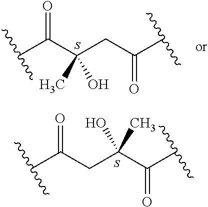 |
| 2-Phenylsuccinic Acid Linker | 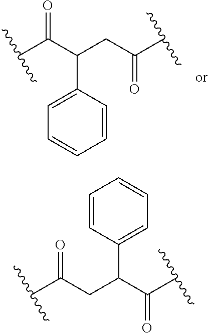 |
| 2,2-Dimethylglutaric Acid Linker | 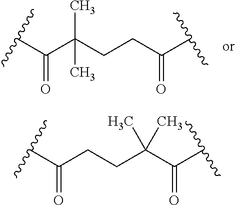 |
| 3,3-Dimethylglutaric Acid Linker | 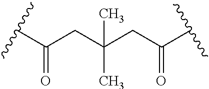 |
| β-Alanine Linker | 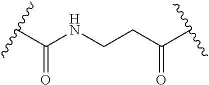 |
| γ-Aminobutyric Acid (GABA) Linker | 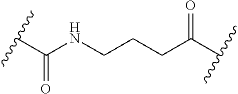 |
| 3-(Carboxy) Butanoic Acid Linker | 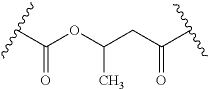 |
| 3-(Carboxy) Propanoic Acid Linker | 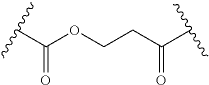 |
| 4-(Carboxy) Butanoic Acid Linker | 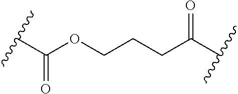 |
| 2-(Carboxy) Propanoic Acid Linker | 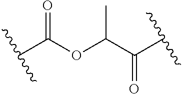 |
| 2-(Carboxy) Acetic Acid Linker | 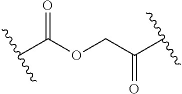 |
| Glutaconic Acid Linker | 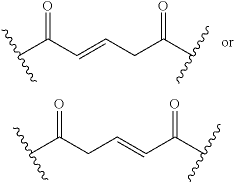 |
| Ketoglutaric Acid Linker | 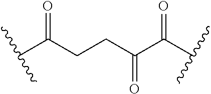 |
| Maleic Acid Linker | 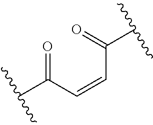 |
| Citraconic Acid Linker | 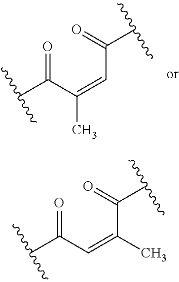 |
| 2,3-Dimethylmaleic Acid Linker | 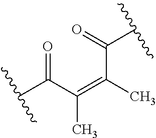 |
| Fumaric Acid Linker | 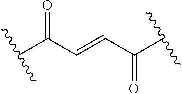 |
| 2,3-Dimethylfumaric Acid Linker | 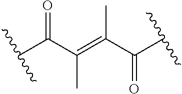 |
| Z-Methoxybutenedioic Acid Linker | 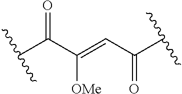 |
| Aconitic Acid Linker | 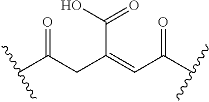 |
| E-Methoxybutenedioc Acid Linker | 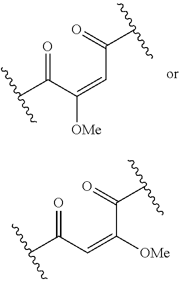 |
| 2-Methylene Glutaric Acid Linker | 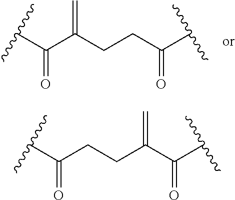 |
| Itaconic Acid Linker | 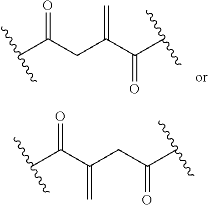 |
| Terephthalic Acid Linker | 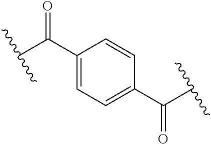 |
| Phthalic Acid Linker | 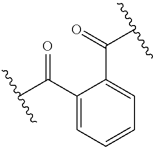 |
| Citroyl Acid Linker | 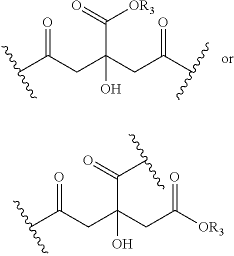 |
| Citric Acid Linker (1) | 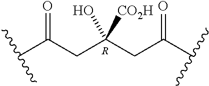 |
| Citric Acid Linker (2) | 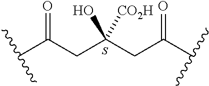 |
| Citric Acid Linker (3) | 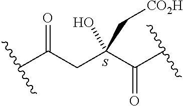 |
| Citric Acid Linker (4) | 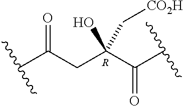 |
| Citric Acid Linker (5) | 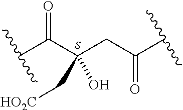 |
| Citric Acid Linker (6) | 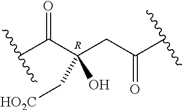 |

In an embodiment, L is

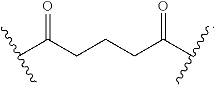

In an embodiment, L is

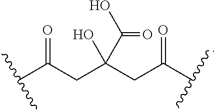

In an embodiment, L is

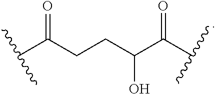

In an embodiment, L is

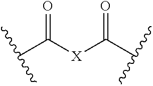

wherein X is a cycloalkyl ring having from 3 to 6 carbon atoms. Preferably, X is cyclopropyl. More preferably, L is cyclopropane-1,2-dicarboxylic acid.

In an embodiment, L is selected from the group comprising:

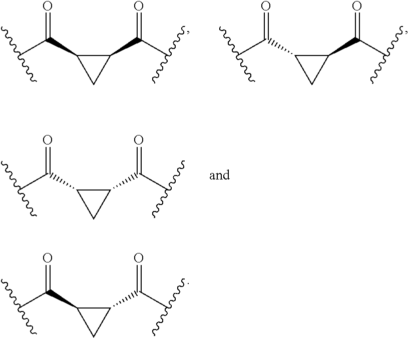

In an embodiment, L is

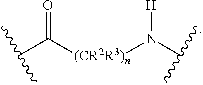

In an embodiment, R² and R³ are each independently selected at each occurance from the group comprising: H, C₁₋₃ alkyl, —OH and —C(═O)R⁶, or R² and R³ together with the atom to which they are bonded form a carbonyl group. Preferably, R⁶ is —OH.

In an embodiment, L is a moiety selected from those recited in the following table:

TABLE 4

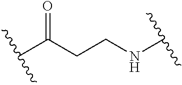

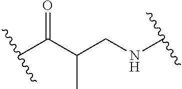

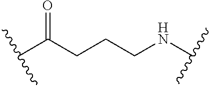

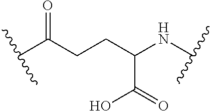

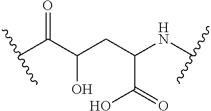

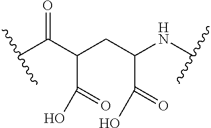

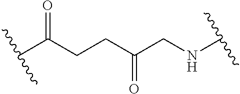

In an embodiment, L is

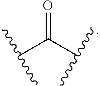

In an embodiment, L is an amino acid residue containing from 2 to 20 carbon atoms. In a preferred embodiment, L is selected from the group comprising: glutamic acid and aspartic acid, preferably L is glutamic acid. In another preferred embodiment, L is β-alanine.

In an embodiment, R is an amino acid residue containing from 2 to 20 carbon atoms. In a further embodiment, R is an amino acid; an amino acid alkyl ester (e.g. an amino acid C₁₋₆ alkyl ester); an N-alkylated amino acid (e.g. a C₁₋₆ N-alkylated amino acid, which can include N-methylcyclopropylated amino acids), preferably the N-alkylated amino acid is an N-methylated amino acid; N,N-dialkylated amino acid (e.g. a C₁₋₆ N,N-dialkylated amino acid, which can include N,N-dimethylcyclopropylated amino acids), preferably the N,N-dialkylated amino acid is an N,N-dimethylated amino acid; an N-acylated amino acid (e.g. a C₁₋₆ N-acylated amino acid); or O-alkylated amino acid (C₁₋₆ O-alkylated amino acid). In N,N-dialkylated amino acids, the alkyl groups may be the same or different.

In an embodiment, R is an amino acid and is selected from the group comprising: valine, N-C₁₋₆ alkylated valine, N,N-C₁₋₆ dialkylated valine, N-methyl valine, N,N-dimethyl valine, alanine, N-C₁₋₆ alkylated alanine, N,N-C₁₋₆ dialkylated alanine, N-methyl alanine, N,N-dimethyl alanine, leucine, N-C₁₋₆ alkylated leucine, N,N-C₁₋₆ dialkylated leucine, N-methyl leucine, N,N-dimethyl leucine, isoleucine, N-C₁₋₆ alkylated isoleucine, N,N-C₁₋₆ dialkylated isoleucine, N-methyl isoleucine and N,N-dimethyl isoleucine.

In an embodiment, R is an amino acid and is selected from the group comprising: glycine, N—C₁₋₆ alkylated glycine, N,N-C₁₋₆ dialkylated glycine, N-methyl glycine, N-methylcyclopropyl glycine, N,N-dimethyl glycine, N,N-dimethylcyclopropyl glycine, alanine, N-C₁₋₆ alkylated alanine, N,N-C₁₋₆ dialkylated alanine, N-methyl alanine, N,N-dimethyl alanine.

In an embodiment, R is a peptide and is selected from the group comprising: serine-glycine, serine-alanine, serine-dimethyl glycine, serine-dimethylcyclopropyl glycine and serine-sarcosine.

In an embodiment, R is a peptide and is selected from the group comprising: threonine-glycine, threonine-alanine, threonine-dimethyl glycine, threonine-dimethylcyclopropyl glycine and threonine-sarcosine.

In an embodiment, R is a peptide having the following amino acid components in which “amino acid 1” is conjugated to the guanfacine end of the conjugate and “amino acid 2” is the terminal amino acid of the peptide:

TABLE 5

| Amino acid 1 | Amino acid 2 |
|---|---|
| S-Serine | Sarcosine |
| S-Serine | Glycine |
| S-Serine | R-Alanine |
| S-Serine | S-Alanine |
| S-Serine | N,N-Dimethylglycine |
| S-Serine | N,N-Dimethylcyclopropane glycine |
| R-Serine | Sarcosine |
| R-Serine | Glycine |
| R-Serine | R-Alanine |
| R-Serine | S-Alanine |
| R-Serine | N,N-Dimethylglycine |
| R-Serine | N,N-Dimethylcyclopropane glycine |
| R-Homoserine | Sarcosine |
| R-Homoserine | Glycine |
| R-Homoserine | R-Alanine |
| R-Homoserine | S-Alanine |
| R-Homoserine | N,N-Dimethylglycine |
| R-Homoserine | N,N-Dimethylcyclopropane glycine |
| S-Homoserine | Sarcosine |
| S-Homoserine | Glycine |
| S-Homoserine | R-Alanine |
| S-Homoserine | S-Alanine |
| S-Homoserine | N,N-Dimethylglycine |
| S-Homoserine | N,N-Dimethylcyclopropane glycine |
| R-Threonine | Sarcosine |
| R-Threonine | Glycine |
| R-Threonine | R-Alanine |
| R-Threonine | S-Alanine |
| R-Threonine | N,N-Dimethylglycine |
| R-Threonine | N,N-Dimethylcyclopropane glycine |
| S-Threonine | Sarcosine |
| S-Threonine | Glycine |
| S-Threonine | R-Alanine |
| S-Threonine | S-Alanine |
| S-Threonine | N,N-Dimethylglycine |
| S-Threonine | N,N-Dimethylcyclopropane glycine |
| R-Allothreonine | Sarcosine |
| R-Allothreonine | Glycine |
| R-Allothreonine | R-Alanine |
| R-Allothreonine | S-Alanine |
| R-Allothreonine | N,N-Dimethylglycine |
| R-Allothreonine | N,N-Dimethylcyclopropane glycine |
| S-Allothreonine | Sarcosine |
| S-Allothreonine | Glycine |
| S-Allothreonine | R-Alanine |
| S-Allothreonine | S-Alanine |
| S-Allothreonine | N,N-Dimethylglycine |
| S-Allothreonine | N,N-Dimethylcyclopropane glycine |

In an embodiment, L is C(═O), and R is serine-glycine, serine-alanine, serine-dimethyl glycine, serine-dimethylcyclopropyl glycine, serine-sarcosine, threonine-glycine, threonine-alanine, threonine-dimethyl glycine, threonine-dimethylcyclopropyl glycine, threonine-sarcosine, homoserine-glycine, homoserine-alanine, homoserine-dimethyl glycine, homoserine-dimethylcyclopropyl glycine, homoserine-sarcosine, allothreonine-glycine, allothreonine-alanine, allothreonine-dimethyl glycine, allothreonine-dimethylcyclopropyl glycine, and allothreonine-sarcosine. In this aspect, the hydroxyl group of the serine, threonine, homoserine, and allothreonine of the R group is attached to the L group.

In an embodiment, L is an amino acid residue containing from 2 to 20 carbon atoms and R is an amino acid residue containing from 2 to 20 carbon atoms. In a preferred embodiment, L is γ-glutamic acid and R is valine. In a preferred embodiment, L is aspartic acid and R is valine. In another preferred embodiment, L is β-alanine and R is valine.

In an embodiment, when L is an amino acid and R is an amino acid, -L-R does not comprise a proteinogenic dipeptide which is conjugated to guanfacine through the alpha carboxylic acid of L. As shown in example 6, proteinogenic dipeptide conjugates which are conjugated to guanfacine through the alpha carboxylic acid of L can be quite unstable under the conditions existing in the GI tract.

In an embodiment, L-R is a conjugate having the following components:

TABLE 6

| L | R |
|---|---|
| β-alanine | R-valine |
| β-alanine | S-valine |
| β-alanine | R-alanine |
| β-alanine | S-alanine |
| β-alanine | R-leucine |
| β-alanine | S-leucine |
| β-alanine | R-isoleucine |
| β-alanine | S-isoleucine |
| γ amino butyric acid | R-valine |
| γ amino butyric acid | S-valine |
| γ amino butyric acid | R-alanine |
| γ amino butyric acid | S-alanine |
| γ amino butyric acid | R-leucine |
| γ amino butyric acid | S-leucine |
| γ amino butyric acid | R-isoleucine |
| γ amino butyric acid | S-isoleucine |
| Aminolevulinic acid | R-valine |
| Aminolevulinic acid | S-valine |
| Aminolevulinic acid | R-alanine |
| Aminolevulinic acid | S-alanine |
| Aminolevulinic acid | R-leucine |
| Aminolevulinic acid | S-leucine |
| Aminolevulinic acid | R-isoleucine |
| Aminolevulinic acid | S-isoleucine |
| β-amino isobutyric acid | R-valine |
| β-amino isobutyric acid | S-valine |
| β-amino isobutyric acid | R-alanine |
| β-amino isobutyric acid | S-alanine |
| β-amino isobutyric acid | R-leucine |
| β-amino isobutyric acid | S-leucine |
| β-amino isobutyric acid | R-isoleucine |
| β-amino isobutyric acid | S-isoleucine |

In an embodiment, L-R is together a dicarboxylic acid-amino acid conjugate having the following components:

TABLE 7

| L | R |
|---|---|
| Glutaric acid | R-valine |
| Glutaric acid | S-valine |
| Glutaric acid | R-alanine |
| Glutaric acid | S-alanine |
| Glutaric acid | R-leucine |
| Glutaric acid | S-leucine |
| Glutaric acid | R-isoleucine |
| Glutaric acid | S-isoleucine |
| Citrate | R-valine |
| Citrate | S-valine |
| Citrate | R-alanine |
| Citrate | S-alanine |
| Citrate | R-leucine |
| Citrate | S-leucine |
| Citrate | R-isoleucine |
| Citrate | S-isoleucine |
| 2-hydroxyglutarate | R-valine |
| 2-hydroxyglutarate | S-valine |
| 2-hydroxyglutarate | R-alanine |
| 2-hydroxyglutarate | S-alanine |
| 2-hydroxyglutarate | R-leucine |
| 2-hydroxyglutarate | S-leucine |
| 2-hydroxyglutarate | R-isoleucine |
| 2-hydroxyglutarate | S-isoleucine |

In an embodiment, L-R is a conjugate having the following components:

| L | R |
|---|---|
| R-γ-glutamic acid | R-valine |
| R-γ-glutamic acid | S-valine |
| R-γ-glutamic acid | R-alanine |
| R-γ-glutamic acid | S-alanine |
| R-γ-glutamic acid | R-leucine |
| R-γ-glutamic acid | S-leucine |
| R-γ-glutamic acid | R-isoleucine |
| R-γ-glutamic acid | S-isoleucine |
| S-γ-glutamic acid | R-valine |
| S-γ-glutamic acid | S-valine |
| S-γ-glutamic acid | R-alanine |
| S-γ-glutamic acid | S-alanine |
| S-γ-glutamic acid | R-leucine |
| S-γ-glutamic acid | S-leucine |
| S-γ-glutamic acid | R-isoleucine |
| S-γ-glutamic acid | S-isoleucine |
| R-β-aspartic acid | R-valine |
| R-β-aspartic acid | S-valine |
| R-β-aspartic acid | R-alanine |
| R-β-aspartic acid | S-alanine |
| R-β-aspartic acid | R-leucine |
| R-β-aspartic acid | S-leucine |
| R-β-aspartic acid | R-isoleucine |
| R-β-aspartic acid | S-isoleucine |
| S-β-aspartic acid | R-valine |
| S-β-aspartic acid | S-valine |
| S-β-aspartic acid | R-alanine |
| S-β-aspartic acid | S-alanine |
| S-β-aspartic acid | R-leucine |
| S-β-aspartic acid | S-leucine |
| S-β-aspartic acid | R-isoleucine |
| S-β-aspartic acid | S-isoleucine |
| γ-carboxy glutamic acid | R-valine |
| γ-carboxy glutamic acid | S-valine |
| γ-carboxy glutamic acid | R-alanine |
| γ-carboxy glutamic acid | S-alanine |
| γ-carboxy glutamic acid | R-leucine |
| γ-carboxy glutamic acid | S-leucine |
| γ-carboxy glutamic acid | R-isoleucine |
| γ-carboxy glutamic acid | S-isoleucine |
| γ-hydroxy glutamic acid | R-valine |
| γ-hydroxy glutamic acid | S-valine |
| γ-hydroxy glutamic acid | R-alanine |
| γ-hydroxy glutamic acid | S-alanine |
| γ-hydroxy glutamic acid | R-leucine |
| γ-hydroxy glutamic acid | S-leucine |
| γ-hydroxy glutamic acid | R-isoleucine |
| γ-hydroxy glutamic acid | S-isoleucine |

In an alternate embodiment, R is a peptide formed from 2 to 10 independently selected amino acids each containing from 2 to 20 carbon atoms.

In an embodiment, R¹ is H.

In an embodiment, R² and R³ are each independently selected at each occurrence from the group comprising: hydrogen, hydroxy, —C(═O)R⁶ and C₁₋₄ alkyl (e.g. —CH₃ or —CH₂CH₃). In an embodiment, R² and R³ are both hydrogen.

In an embodiment, R⁴ and R⁵ are each independently selected from the group comprising: H and C₁₋₄ alkyl.

In an embodiment, R⁶ is —OH.

In an embodiment, R is an amino acid residue containing from 2 to 20 carbon atoms and L is

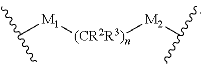

In an embodiment, R is an amino acid residue containing from 2 to 20 carbon atoms and L is

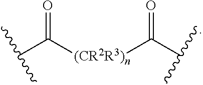

In an embodiment, L is

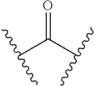

and R is a peptide formed from 2 to 10 independently selected amino acids each containing from 2 to 20 carbon atoms. In the context of this invention, the term ‘amino acid residue’ means an amino acid, an amino acid alkyl ester, an amino acid aryl ester, an N-alkylated amino acid (e.g. a mono- or di-N-methylated amino acid), an N-acylated amino acid, an N-arylated amino acid, an N-alkylated amino acid ester, an N-acylated amino acid ester, an N-arylated amino acid ester, an O-alkylated amino acid, an O-arylated amino acid, an O-acylated amino acid, an O-alkylated amino acid ester, an O-arylated amino acid ester, an O-acylated amino acid ester, an S-alkylated amino acid, an S-acylated amino acid, an S-arylated amino acid, an S-alkylated amino acid ester, an S-acylated amino acid ester or an S-arylated amino acid ester. In other words, the invention also envisages amino acid derivatives such as those mentioned above which have been functionalized by simple synthetic transformations known in the art (e.g. as described in “Protective Groups in Organic Synthesis” by T W Greene and P G M Wuts, John Wiley & Sons Inc (1999), and references therein.

In the context of this invention, the term ‘amino acid’ includes both natural amino acids (including proteinogenic amino acids) and non-natural amino acids. The term “natural amino acid” may also include in addition other amino acids which can be incorporated into proteins during translation (including pyrrolysine, ornithine and selenocysteine). An amino acid generally has the Formula:

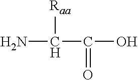

wherein Rₐₐ is referred to as the amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine. The invention also contemplates the use of homologues of natural amino acids such as, but not limited to, homoarginine. The invention also contemplates the use of beta amino acids such as, but not limited to, beta alanine. The invention also contemplates the use of certain lactam analogues of natural amino acids such as, but not limited to, pyroglutamine.

In an embodiment, the guanfacine prodrug of the present invention is a conjugate containing one or more amino acid residues and is optionally separated from the guanfacine portion by a linking group. Each amino acid may independently be linked to its neighbour via the carboxyl group of the amino acid, be linked via the side chain of the amino acid which itself may for example contain a carbonyl, amino, or thio group, or may be linked via its amino group. The first amino acid residue may be bonded to the guanidino group of guanfacine via the carboxyl group of the amino acid or via functionality present on the side chain of the amino acid.

In an embodiment, the guanfacine prodrug of the present invention is a conjugate containing a single amino acid which is separated from the guanfacine portion by a linking group.

In an embodiment, when L is absent, R is a peptide formed from 2 to 10 independently selected amino acids each containing from 2 to 20 carbon atoms.

In another aspect of the present invention, there is provided a prodrug of an active guanfacine metabolite of Formula (III), or a pharmaceutically acceptable salt or tautomer thereof:

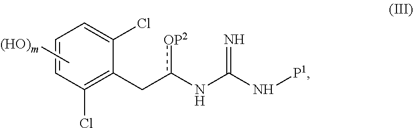

wherein:

P¹ is hydrogen or -L-R;

P² is absent, hydrogen or -L-R;

provided that when P¹ is hydrogen, P² is not absent;

L is absent, or a group selected from the group comprising:

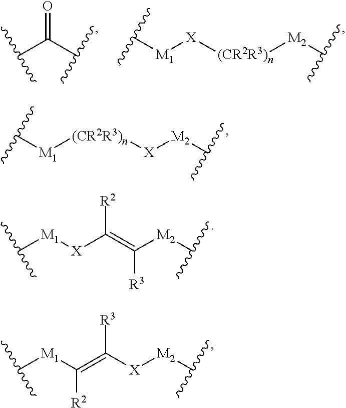

an amino acid residue containing from 2 to 20 carbon atoms, and a peptide formed from 2 to 10 independently selected amino acids each containing from 2 to 20 carbon atoms; wherein:

M₁ is absent or is selected from the group comprising: —CH₂—,

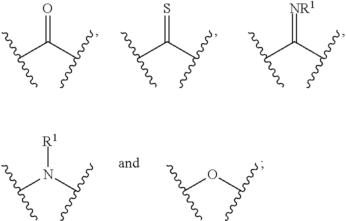

wherein R¹ is selected from the group comprising: H, C₁₋₄ alkyl and C₃₋₈ cycloalkyl;

M₂ is absent or is selected from the group comprising: —CH₂—,

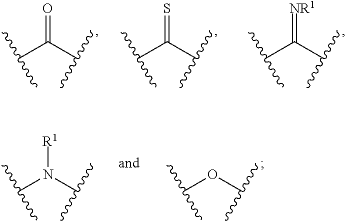

wherein R¹ is selected from the group comprising: H, C₁₋₄ alkyl and C₃₋₈ cycloalkyl;

R² and R³ are each independently selected at each occurrence from the group comprising: hydrogen, hydroxy, C₁₋₆ alkoxy, C₁₋₆ alkyl C₁₋₆ alkoxy, —(CR⁴R⁵)ₙOC(═O)R⁶, —(CR⁴R⁵)ₙC(═O)R⁶, —C(═O)R⁶, C₁₋₆ alkyl, C₁₋₆ haloalkyl, aryl, —N R⁴R⁵ and —NR⁴(CO)R⁶; or together with the atom to which they are bonded, R² and R³ may form a carbonyl, an ethylene or a C₃₋₆ cycloalkyl;

R⁴ and R⁵ are each independently selected from the group comprising: H, C₁₋₆ alkyl, C₁₋₆ haloalkyl, C₃₋₈ cycloalkyl and phenyl;

R⁶ is selected from the group comprising: hydroxyl, C₁₋₆ alkyl, C₁₋₆ alkoxy, C₃₋₈ cycloalkyl and phenyl;

X is selected from the group comprising: a bond, —O—, —NH—, —CR²R³— and a saturated or unsaturated ring having from 3 to 6 carbon atoms in the ring;

R is hydroxy, an amino acid residue containing from 2 to 20 carbon atoms or a peptide formed from 2 to 10 independently selected amino acids each containing from 2 to 20 carbon atoms, or R is a group selected from the group comprising: —NH₂ and —NR⁴R⁵;

n is at each occurance independently an integer of 0-16; and

m is an integer of 1-3.

In another aspect, the present invention provides a method of treating a disorder in a subject in need thereof with guanfacine. The method comprises orally administering an effective amount of a guanfacine prodrug of the present invention to the subject. The disorder may be one treatable with guanfacine. For example, the disorder may be attention deficit hyperactivity disorder (ADHD). An alternative psychiatric condition treatable with guanfacine is oppositional defiance disorder (ODD). Alternatively, the disorder may be a cardiovascular condition such as hypertension. The disorder may also be a disorder selected from the group comprising: neuropathic pain, cognitive impairment associated with schizophrenia (CIAS), anxiety (including PTSD, OCD, self injury), addiction withdrawal and autism. The disorder may also be chemotherapy induced mucositis. The disorder may also be post traumatic stress syndrome. Alternatively, the disorder may be characterized by the patient suffering from hot flushes.

In another aspect, the present invention provides a guanfacine conjugate of the present invention for use in the treatment of attention deficit hyperactivity disorder (ADHD), oppositional defiance disorder (ODD), a cardiovascular condition such as hypertension, neuropathic pain, cognitive impairment associated with schizophrenia (CIAS), anxiety (including PTSD, OCD, self injury), addiction withdrawal, autism, chemotherapy induced mucositis, post traumatic stress syndrome or a disorder characterized by hot flushes.

In one embodiment, there is provided a method of reducing adverse gastrointestinal side effects associated with guanfacine treatment in a mammal. The method includes
(a) forming a guanfacine prodrug of Formula (I) or a pharmaceutically acceptable salt thereof; and
(b) administering the prodrug or a pharmaceutically acceptable salt thereof to a mammal in need thereof. Typically, the mammal is a human subject.

The guanfacine prodrugs described herein induce statistically significant lower average (e.g., mean) effects on gut motility in the gastrointestinal environment as compared to a non-prodrug guanfacine salt form such as guanfacine HCl.

In an alternative aspect of the invention, a method for improving the pharmacokinetics and extending the duration of action of guanfacine in a subject in need thereof is provided. The method comprises administering to a subject in need thereof an effective amount of a prodrug of the present invention, or a composition thereof, wherein the plasma concentration time profile is modulated to minimize an initial upsurge in concentration of guanfacine, minimizing any unwanted cardiovascular or somnolent effects, while significantly extending the time for which the drug persists in plasma (resulting from continuing generation from the prodrug) and hence duration of action.

In a further aspect, a method for reducing inter- or intra-subject variability of guanfacine plasma levels is provided. The method comprises administering to a subject, or group of subjects in need thereof, an effective amount of a prodrug of the present invention, or a composition thereof.

In one preferred embodiment, the present invention is directed to a method for minimizing gastrointestinal side effects such as constipation normally associated with administration of guanfacine. The method comprises orally administering a guanfacine prodrug or pharmaceutically acceptable salt of the present invention, and wherein upon oral administration, the prodrug or pharmaceutically acceptable salt minimizes, if not completely avoids, the gastrointestinal side effects usually seen after oral administration of the unbound guanfacine. The amount of guanfacine is preferably a therapeutically effective amount.

The present invention relates to natural and/or non-natural amino acids and short-chain peptides of guanfacine which preclude interaction between the α-2 adrenoceptors located in the gut and the active drug, so minimizing the risk of constipation. In addition, the prodrugs provided herein deliver a pharmacologically effective amount of the drug to treat various psychiatric and/or cardiovascular conditions. Such use of prodrugs of guanfacine reduces intra- and inter-subject variability in plasma concentration and so provides consistent therapeutic efficacy. Additionally, the presence of quantities of unhydrolyzed prodrug in tissue compartments and/or plasma may provide a reservoir for continued generation of the active drug. Continued generation of guanfacine maintains plasma drug levels, thereby reducing the frequency of drug dosage. These benefits would be expected to improve patient compliance.

These and other embodiments are disclosed or are apparent from and encompassed by the following Detailed Description.

### BRIEF DESCRIPTION OF THE DRAWINGS

FIG. 1 illustrates plasma concentration profiles for guanfacine following administration of guanfacine or compound 1 to primates at 0.5 mg/kg guanfacine free base equivalents.

FIG. 2 illustrates plasma concentration profiles for guanfacine following administration of guanfacine or compound 2 to primates at 0.5 mg/kg guanfacine free base equivalents.

FIG. 3 illustrates plasma concentration profiles for guanfacine following administration of guanfacine or compound 5 to primates at 0.5 mg/kg guanfacine free base equivalents.

FIG. 4 illustrates plasma concentration profiles for guanfacine following administration of guanfacine or compound 61 to primates at 0.5 mg/kg guanfacine free base equivalents.

FIG. 5 illustrates plasma concentration profiles for guanfacine following administration of guanfacine or compound 63 to primates at 0.5 mg/kg guanfacine free base equivalents.

### DETAILED DESCRIPTION OF THE INVENTION

### A. Definitions

As used herein:

The term “peptide” refers to an amino acid chain consisting of 2 to 9 amino acids (bound via peptide bonds), unless otherwise specified. In preferred embodiments, the peptide used in the present invention is 2 or 3 amino acids in length. The present invention also concerns branched peptides, where an amino acid can be bound to another amino acid's side chain.

An amino acid is a compound represented by NH₂—CH(Rₐₐ)—COOH, wherein Rₐₐ is an amino acid side chain (e.g., when Rₐₐ═H, the amino acid is glycine). The term amino acid side chain, as used herein, is the substituent on the alpha-carbon of an amino acid.

The amino acids contemplated for use in the prodrugs of the present invention include both natural and non-natural amino acids. In one preferred embodiment, the amino acids are natural amino acids. In an embodiment, the natural amino acids are proteinogenic amino acids. The side chains Rₐₐ can be in either the (R) or the (S) configuration. Both L- and D-amino acids are within the scope of the present invention.

The term ‘amino acid’ includes both natural amino acids and non-natural amino acids. A “natural amino acid” includes the twenty amino acids used for protein biosynthesis (proteinogenic amino acids) as well as other amino acids which can be incorporated into proteins during translation (including pyrrolysine, ornathine and selenocysteine). A natural amino acid generally has the formula

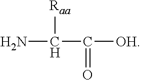

Rₐₐ, is referred to as the amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine and homologues thereof.

Examples of natural amino acid sidechains include —H (glycine), —CH₃ (alanine), —CH(CH₃)₂ (valine), —CH(CH₃)CH₂CH₃ (isoleucine), —CH₂CH(CH₃)₂ (leucine), —CH₂C₆H₅ (phenylalanine), —CH₂C₆H₄-p-OH (tyrosine), —CH₂OH (serine), —CH(OH)CH₃ (threonine), —CH₂-3-indolyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-natural amino acid” is an organic compound which is an amino acid, but is not among those encoded by the standard genetic code, or incorporated into proteins during translation. Non-natural amino acids, thus, include amino acids or analogs of amino acids other than the 20 naturally-occurring amino acids and include, but are not limited to, the D-isostereomers of amino acids. Examples of non-natural amino acids include, but are not limited to: citrulline, homocitrulline, hydroxyproline, homoarginine, homoserine, homotyrosine, homoproline, ornithine, 4-amino-phenylalanine, sarcosine, biphenylalanine, homophenylalanine, 4-am ino-phenylalanine, 4-nitro-phenylalanine, 4-fluoro-phenylalanine, 2,3,4,5,6-pentafluoro-phenylalanine, norleucine, cyclohexylalanine, α-aminoisobutyric acid, N-methyl-alanine, N-methyl-glycine, N-methyl-glutamic acid, tert-butylglycine, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine, dipropylglycine and derivatives thereof wherein the amine nitrogen has been mono- or di-alkylated.

The amino acids contemplated by the present invention also include metabolites of the natural amino acids including, but not limited to, N-acetyl cysteine, N-acetyl serine, and N-acetyl threonine.

The term “polar amino acid” refers to a hydrophilic amino acid having a polar side chain. The polar amino acid can be positively or negatively charged, or neutral at physiological pH, but the polar side chain has at least one bond in which the pair of electrons shared in common by two atoms is held more closely by one of the atoms. Genetically encoded polar amino acids include Arg (R), Asp (D), Glu (E), Histidine (H), Lysine (K), Asn (N), Gln (Q) Ser (S) and Thr (T). The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl refers to a straight or branched alkyl containing at least 1, and at most 10, carbon atoms. Examples of “alkyl” as used herein include, but are not limited to, methyl, ethyl, n-propyl, n-butyl, n-pentyl, i-butyl, i-propyl, t-butyl, hexyl, heptyl, octyl, nonyl and decyl. Preferably, the alkyl group is a lower alkyl of from about 1 to 7 carbons, yet more preferably about 1 to 4 carbons. The alkyl group can be substituted or unsubstituted.

The term “acyl” refers to the group —C(═O)R⁶ wherein R⁶ is C₁₋₆ alkyl.

The term “substituted alkyl” as used herein denotes alkyl radicals wherein at least one hydrogen is replaced by one or more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition is pertinent whether the term is applied to a substituent itself or to a substituent of a substituent.

The term “heterocycle” refers to a stable 3- to 1 5-membered ring radical which consists of carbon atoms and from one to five heteroatoms selected from nitrogen, phosphorus, oxygen and sulfur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition is pertinent whether the term is applied to a substituent itself or to a substituent of a substituent.

The term “alkoxy” refers to an alkyl group of an indicated number of carbon atoms attached to the parent molecular moiety through an oxygen bridge. Examples of alkoxy groups include, for example, methoxy, ethoxy, propoxy and isopropoxy. When the term “alkoxy” is used without reference to a number of carbon atoms, it is to be understood to refer to a C₁-C₁₀ alkoxy in which the alkyl group can be straight, branched, saturated or unsaturated alkyls containing at least 1, and at most 10, carbon atoms. Preferably, it is a lower alkoxy of from about 1 to 4 carbons.

The term “carbonyl” refers to a group —C(═O).

The term “carboxyl” refers to a group —CO₂H and consists of a carbonyl and a hydroxyl group (More specifically, C(═O)OH).

“Dicarboxylate linker group,” “dicarboxylic acid linker,” and “dicarboxylate,” are synonymous, and refer to the group —C(═O)—[CR¹R²]ₙC(═O)— in the moiety

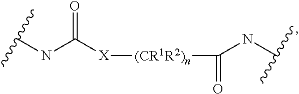

wherein N at one end is present in the unbound form of guanfacine, N at the other end is the nitrogen of the N terminus of a peptide, or nitrogen of the amino group of an amino acid, (n) is an integer of from about zero to about 9, preferably about 2. Prodrug moieties described herein may be referred to based on their amino acid or peptide and the dicarboxylate linker group. The amino acid or peptide in such a reference should be assumed to be bound via an amino terminus on the amino acid or peptide to one carboxyl group of the dicarboxylic acid, while the other carboxyl group is attached to guanfacine. The dicarboxylate linker group may or may not be variously substituted as stipulated earlier.

The term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, tetrahydronaphthalene and biphenyl. Preferred examples of aryl groups include phenyl.

The term “halo” or “halogen” refers to fluoro, chloro, bromo, and iodo.

The term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in humans.

A “pharmaceutically acceptable excipient” means an excipient that is useful in preparing a pharmaceutical composition that is generally safe, non-toxic and neither biologically nor otherwise undesirable, and includes an excipient that is acceptable for human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

The term “subject” refers to humans.

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be amelioration of the typical symptoms of ADHD. In further and/or alternative embodiments, the therapeutic response will be amelioration of the typical symptoms of opposition defiance disorder (ODD), hypertension, pain (neuropathic pain), cognitive impairment in psychosis, cognitive impairment associated with schizophrenia (CIAS), post traumatic stress disorder (PTSD), anxiety (including PTSD, OCD, self injury), addiction withdrawal, autism, hot flushes, chemotherapy-induced mucositis, etc. It is further within the competency of one skilled in the art to determine appropriate treatment duration, appropriate doses, and any potential combination treatments, based upon an evaluation of therapeutic response.

“Reducing gastrointestinal side effects associated with guanfacine therapy” shall be understood to mean a reduction, amelioration and/or prevention of the occurrence of gastrointestinal side effects (e.g., constipation) realized in patients treated with the prodrug described herein as compared to patients which have received a non-prodrug guanfacine salt in an immediate release or sustained release form. Reduction of gastrointestinal side effects is deemed to occur when a patient achieves positive clinical results. For example, successful reduction of gastrointestinal side effects shall be deemed to occur when at least about 10% (i.e. at least about 15%) or preferably at least about 20%, more preferably at least about 30% or higher (i.e., about 40%, 50%) decrease in constipation including other clinical markers contemplated by the artisan in the field is realized when compared to that observed in the treatment with a non-prodrug guanfacine. In certain aspects, successful reduction of gastrointestinal side effects can be determined by changes in gut motility induced by the prodrug described herein as compared to a non-prodrug guanfacine salt in an immediate release or sustained release form. In this aspect, statistical significance relative to a non-prodrug guanfacine can be at least about 0.058, and preferably <0.001.

The term “at least about” comprises the numbers equal to or larger than the numbers referred to. In various embodiments, such as when referring to the decrease in gut motility, the term “at least about 15%” includes the terms “at least about 16%”, “at least about 17%”, at least about 18%” and so forth. Likewise, in some embodiments, the term “at least about 30%” includes the terms “at least about 31%”, “at least about 32%”, and so forth.

The term “active ingredient,” unless specifically indicated, is to be understood as referring to the guanfacine portion of the prodrug, as described herein.

The term “salts” can include acid addition salts or addition salts of free bases. Suitable pharmaceutically acceptable salts (for example, of the carboxyl terminus of the amino acid or peptide) include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate (see, for example, Berge, et al. “Pharmaceutical Salts,” J. Pharm. Sci. 1977;66:1).

The term “about,” unless otherwise indicated, refers to ±10% of the given value.

The present invention also includes the synthesis of all pharmaceutically acceptable isotopically-labelled compounds of Formula (I) wherein one or more atoms are replaced by atoms having the same atomic number, but an atomic mass or mass number different from the atomic mass or mass number most commonly found in nature.

Substitution with stable isotopes such as deuterium, i.e. ²H, may afford certain therapeutic advantages resulting from greater metabolic stability, for example, increased in vivo half-life or reduced dosage requirements, and hence may be preferred in some circumstances.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

B. Advantages of the Guanfacine Prodrugs of the Present Invention

The use of the guanfacine prodrugs of the present invention provides a means of delivering guanfacine to the systemic circulation but avoiding direct contact between the active drug and α-2-adrenoceptors in the GI tract so minimizing any potential constipating effects. It is possible that part of the constipating actions of α-2-adrenoceptors may be elicited directly within the gut. Reduction of the adverse GI side-effects associated with administration may be a particular advantage of using a prodrug of the present invention.

Preferably, guanfacine therapy with the prodrugs described herein, when administered orally, induces significantly lower average (i.e. mean) effects on gut motility in the gastrointestinal environment of the patient than a non-prodrug guanfacine salt form such as guanfacine hydrochloride salt.

Without wishing to be bound to any particular theory, it is believed that the amino acid or peptide portion of the guanfacine prodrugs selectively exploits the inherent di- and tripeptide transporter Pept1 within the digestive tract. Once absorbed, these prodrugs may provide a reservoir from which the active drug species may continue to be generated simulating the delivery from a sustained release preparation. This approach avoids the need for enteric coated sustained release formulations which may be subject to premature coat erosion in the stomach due to the presence of food. Using a prodrug provides an alternate means of continuous delivery since it is believed that drug is released from the amino acid or peptide prodrug by hepatic and extrahepatic hydrolases which are, in part, present in red blood cells and/or plasma. Alternatively, the prodrug may be metabolized to an intermediate which may be converted to the active drug through chemical or enzymatic processes.

Additionally, the use of the prodrugs of the present invention can provide greater consistency in response as the result of more consistent oral bioavailability. As a result of this consistent oral bioavailability, the prodrugs of the present invention offer a significant reduction of inter- and intrasubject variability of guanfacine plasma and CNS concentrations and, hence, significantly less fluctuation in therapeutic response for a single patient, or among a patient population providing improved patient benefit.

### C. Methods of Treatment

The present invention provides a method for treating a disorder in a subject in need thereof with guanfacine. The method comprises orally administering an effective amount of a guanfacine prodrug of the present invention to the subject. The disorder may be one treatable with guanfacine. For example, the disorder may be psychiatric conditions such as attention deficit hyperactivity disorder or oppositional defiance disorder. The prodrug can be any guanfacine prodrug encompassed by Formula (I).

The present invention also provides a guanfacine conjugate of Formula (I) for use in the treatment of a psychiatric condition such as attention deficit hyperactivity disorder or oppositional defiance disorder.

In one aspect, the present invention is directed to a method for minimizing the gastrointestinal side effects normally associated with administration of guanfacine. The method comprises orally administering a guanfacine prodrug or pharmaceutically acceptable salt of the present invention, and wherein upon oral administration, the prodrug or pharmaceutically acceptable salt minimizes, if not completely avoids, the constipating effects frequently seen after administration of higher oral doses of the unbound guanfacine. The amount of guanfacine is preferably a therapeutically effective amount. The prodrug can be any guanfacine prodrug encompassed by Formula (I).

In view of the above, there are provided methods of reducing gastrointestinal side effects associated with guanfacine therapy in a mammal. The methods include:
(a) forming a guanfacine prodrug of Formula (I) or a pharmaceutically acceptable salt thereof; and
(b) administering the prodrug or a pharmaceutically acceptable salt thereof to a mammal in need thereof.

In another aspect, the invention provides a method of treating an attention deficit hyperactivity disorder in a mammal. The method includes administering a prodrug of Formula (I) or a pharmaceutically acceptable salt thereof to a mammal in need thereof.

The present invention also provides a guanfacine conjugate of Formula (I) for use in the treatment of attention deficit hyperactivity disorder in a mammal.

In yet another aspect, the invention provides a method of treating hypertension in a mammal. The method is conducted by administering a prodrug of Formula (I) or a pharmaceutically acceptable salt thereof to a mammal in need thereof.

The present invention also provides a guanfacine conjugate of Formula (I) for use in the treatment of hypertension in a mammal.

Ideally, the prodrugs employed in the methods described herein, when administered orally, should achieve therapeutically effective guanfacine plasma concentration. In one embodiment, the prodrugs employed in the method described herein include guanfacine attached to valine.

In one preferred embodiment, the prodrugs of Formula (I) or the pharmaceutically acceptable salts thereof are orally administered. In some preferred embodiments, the method protocol includes administering the prodrugs of Formula (I) or the pharmaceutically acceptable salts thereof in a daily amount of from about 1 mg to about 100 mg, preferably from about 1 mg to about 50 mg, more preferably from about 1 mg to about 15 mg, more preferably from about 1 mg to about 10 mg and more preferably from about 1 mg to about 5 mg based on the amount of guanfacine in free base form. If the systemic availability from the prodrug yields a lower absolute oral bioavailablity, then the preferred dosage is from about 2 mg to about 10 mg.

In all aspects of the invention where the conjugate of Formula (I) or the pharmaceutically acceptable salt thereof is administered, the dosage mentioned is based on the amount of guanfacine free base rather than the amount of the conjugate administered.

The present method is useful for, among other things, avoiding the constipating effects associated with guanfacine administration resulting from α-2 adrenoceptor mediated inhibition of gut motility as compared to a treatment with guanfacine in non-prodrug salt form.

Alternatively, the present invention provides a method for improving the pharmacokinetics of guanfacine in a subject in need thereof. The method comprises administering to a subject in need thereof an effective amount of a prodrug of the present invention, or a composition thereof, wherein the rate and consistency of delivery of guanfacine provided by the prodrug offers advantage over that seen when guanfacine in a non-prodrug form is administered alone. These benefits include a modulation of the attainment of Cₘₐₓ so minimizing unwanted cardiovascular effects, greater consistency in attainment of plasma levels and thereby therapeutic response and prolonged maintenance of plasma drug levels reducing dosing frequency and improving patient compliance. The prodrug can be any guanfacine prodrug encompassed by Formula (I).

In a further alternative aspect, the present invention provides a method of reducing effects of guanfacine on gut motility. The method includes the steps of
(a) reacting guanfacine with an activated amino acid (e.g. glutamic acid) capable of forming a covalent bond with the guanfacine under conditions effective to form a prodrug of Formula (I) and
(b) administering the prodrug of Formula (I) or the pharmaceutically acceptable salt thereof to a mammal in need thereof.

The present invention also provides a guanfacine conjugate of Formula (I) for use in the reduction of the effects of guanfacine on gut motility.

### D. Salts, Solvates, & Derivatives of the Compounds of the Invention

The methods of the present invention further encompass the use of salts and solvates of the guanfacine prodrugs described herein. In one embodiment, the invention disclosed herein is meant to encompass all pharmaceutically acceptable salts of guanfacine prodrugs (including those of the carboxyl terminus of the amino acid as well as those of the basic nitrogens).

Typically, a pharmaceutically acceptable salt of a prodrug of guanfacine used in the practice of the present invention is prepared by reaction of the prodrug with an acid or base as appropriate. The salt may precipitate from solution and be collected by filtration or may be recovered by evaporation of the solvent in accordance with methods well known to those skilled in the art.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metal bases or amines, such as alkali and alkaline earth metal hydroxides or organic amines. Examples of metals used as cations are sodium, potassium, magnesium, calcium, and the like. Examples of suitable amines are N,N′-d ibenzylethylenediam ine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, for example, Brittain. Polymorphism in Pharmaceutical solids. Marcel Decker, New York, 1999.). The compounds useful in the practice of the present invention can have one or more chiral centers and, depending on the nature of individual components, they can also have geometrical isomers.

### E. Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier or excipient selected with regard to the intended route of administration and standard pharmaceutical practice. The compositions of the present invention also include pharmaceutically acceptable salts of the guanfacine prodrugs, as described above.

While it is anticipated that the formulations of the invention may be immediate-release dosage forms, i.e., dosage forms that release the prodrug at the site of absorption immediately, in an alternative embodiment, the prodrugs described herein can be as part of controlled-release formulation, i.e. dosage forms that release the prodrug over a predetermined period of time. Controlled release dosage forms may be of any conventional type, e.g. in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20^{th} Edition, 2000, pp. 858-914.

However, since absorption of amino acid and peptide prodrugs of guanfacine may proceed via an active transporter such as Pept1, unconventional controlled dosage forms may be desirable. As the Pept1 transporter is believed to be largely confined to the upper GI tract, this may limit the opportunity for continued absorption along the whole length of the GI tract. For those prodrugs of guanfacine which do not result in sustained plasma drugs levels due to continuous generation of active from a systemic reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption takes place and effectively prolonging plasma drug levels. Other drug delivery systems affording delayed progression along the GI tract may also be of value.

The formulations of the present invention can be administered from one to six times daily, depending on the dosage form and dosage.

In one aspect, the present invention provides a pharmaceutical composition containing at least one active pharmaceutical ingredient (i.e., a guanfacine prodrug), or a pharmaceutically acceptable derivative (e.g., a salt or solvate) thereof, and a pharmaceutically acceptable carrier or other excipient. In particular, the invention provides a pharmaceutical composition including a therapeutically effective amount of at least one prodrug described herein, or a pharmaceutically acceptable derivative thereof, and a pharmaceutically acceptable carrier or excipient.

For the methods of the invention, the prodrug employed in the present invention may be used in combination with other therapies and/or active agents. Accordingly, the present invention provides, in a further aspect, a pharmaceutical composition including at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds must be stable and compatible with each other and the other components of the formulation. When formulated separately the compounds may be provided in any convenient formulation, conveniently in such manner as is known for such compounds in the art.

The prodrugs used herein may be formulated for administration in any convenient way for use in human medicine and the invention therefore includes within its scope pharmaceutical compositions comprising a compound of the invention adapted for use in human medicine. Such compositions may be presented for use in a conventional manner with the aid of one or more pharmaceutically acceptable excipients or carriers. Acceptable carriers and excipients for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example, see International Patent Application No. WO 02/00196 (SmithKline Beecham).

The prodrugs and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, and propyl paraben).

Suitable examples of pharmaceutically acceptable stabilizers and antioxidants include, but are not limited to, ethylenediaminetetra-acetic acid (EDTA), thiourea, tocopherol and butyl hydroxyan (hydroxyanisole).

The pharmaceutical compositions of the invention may contain from 0.01 to 99% weight per volume of the prodrugs encompassed by the present invention.

### F. Doses

The doses described throughout the specification refer to the amount of guanfacine in the composition, in free base form.

Appropriate patients (subjects) to be treated according to the methods of the invention include any human in need of such treatment. Methods for the diagnosis and clinical evaluation of ADHD or ODD including the severity of the condition experienced by a human are well known in the art. Thus, it is within the skill of the ordinary practitioner in the art (e.g., a medical doctor) to determine if a patient is in need of treatment.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In a preferred embodiment, an effective amount of prodrugs of Formula (I) is from about 1 mg to about 100 mg, preferably from about 1 to about 50 mg, and more preferably from about 1 mg to about 5 mg. If the prodrugs of Formula (I) provide near complete oral bioavailability, the preferred dosage is from about 1 to about 5 mg , based on the currently effective maximum daily doses of from about 1 to about 5 mg. If the systemic availability from the prodrug yields a lower absolute oral bioavailablity, then the preferred dosage is from about 2 mg to about 10 mg. The prodrugs, as described herein, may be administered once daily or divided into multiple doses as part of multiple dosing treatment protocol.

Depending on the severity of the condition to be treated, a suitable therapeutically effective and safe dosage, as may readily be determined within the skill of the art, and without undue experimentation, may be administered to subjects. For oral administration to humans, the daily dosage level of the prodrug may be in single or divided doses. The duration of treatment may be determined by one of ordinary skill in the art, and should reflect the magnitude of the condition.

In the methods of treating ADHD/ODD or hypertension, the prodrugs encompassed by the present invention may be administered in conjunction with other therapies and/or in combination with other active agents. For example, the prodrugs encompassed by the present invention may be administered to a patient in combination with other active agents used in the management of these conditions. An active agent to be administered in combination with the prodrugs encompassed by the present invention may include, for example, a drug selected from the group consisting of stimulant drugs such as amphetamine or methyl phenidate or non stimulant agents such atomoxetine. In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating ADHD/ODD or hypertension, the dose of each compound may differ from that when the compound is used alone. Appropriate doses will be readily appreciated by those skilled in the art.

### G. Synthesis of the Prodrugs

Generally, the methods of preparing prodrugs described herein include reacting guanfacine with an activated amino acid or peptide under conditions effective to form prodrugs of Formula (I). Activated amino acids useful in the methods described herein can be prepared by standard techniques known to those of ordinary skill, for example, coupling a dipeptide with N-hydroxysuccinimide (NHS) to prepare an NHS ester, reacting an amino acid with phosgene to prepare isocyanate, or extending an amino acid with a dicarboxylic acid which can be activated as an NHS ester. The methods provide a guanfacine prodrug where guanfacine is bonded to a dipeptide through an amide linkage, to a dipeptide through a carbamate linkage, to an amino acid through urea linkage, or to an amino acid through a dicarboxylic acid linker forming an amide linkage.

For purposes of illustration, the methods of preparing prodrugs described herein include:
(a) reacting an activated amino acid and peptide having the formula;

LG-L₁-Rₐ-PG

with an amino group of guanfacine under basic conditions sufficient to form a protected guanfacine prodrug having the formula:

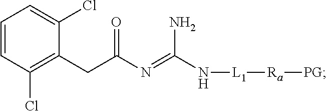

and
(b) deprotecting the guanfacine prodrug with an acid to form a prodrug of Formula (I):

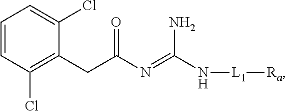

wherein
L₁ is an amino acid, a carbonyl or a dicarboxylic acid;
Rₐ is an amino acid or a peptide,
LG is a leaving group; and
PG is a protecting group such as BOC and t-Bu.

The leaving group useful in the preparation includes NHS or p-nitrophenyloxy and other leaving groups known by those of ordinary skill in the art.

It will be understood that other art recognized protecting groups can be used in place of BOC and t-Bu.

Preferably, the reactions are carried out in an inert solvent such as 1,2-dimethoxyethane (DME), ethyl acetate, methanol, methylene chloride, chloroform, N,N′-dimethylformamide (DMF) or mixtures thereof. The reactions can be preferably conducted in the presence of a base, such as N-methylmorpholine (NMM), dimethylaminopyridine (DMAP), diisopropylethylamine, pyridine, triethylamine, etc. to neutralize any acids generated. The reactions can be carried out at a temperature from about 0° C. up to about 22° C. (room temperature).

### EXAMPLES

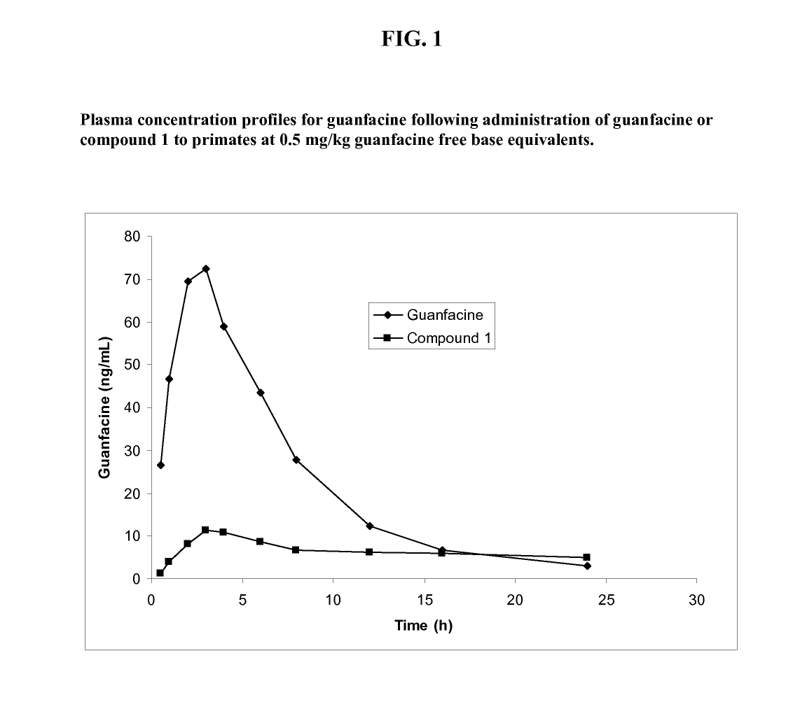

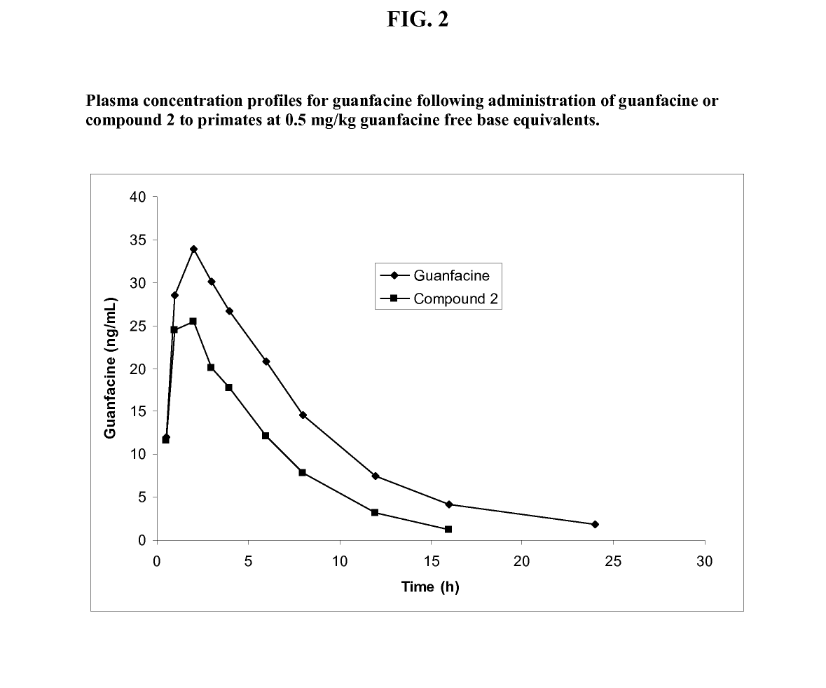

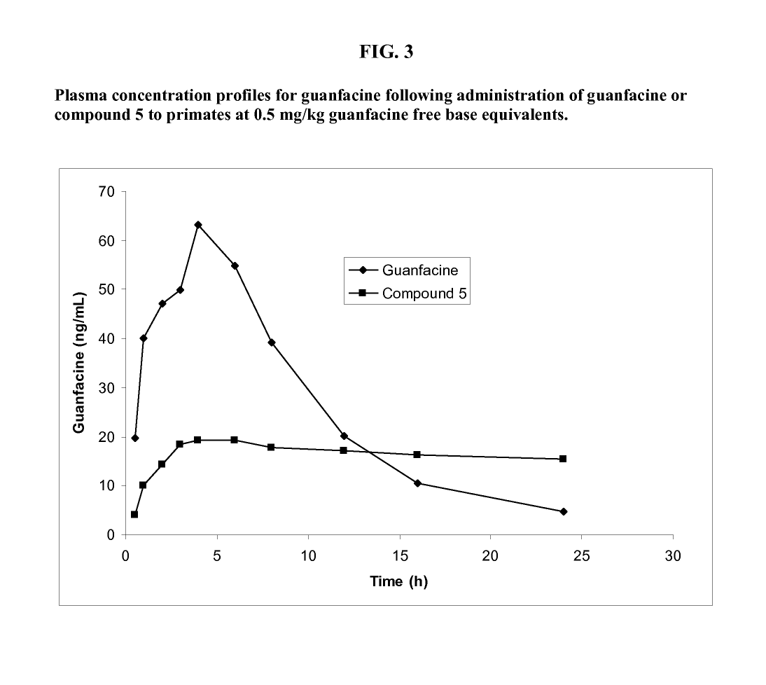

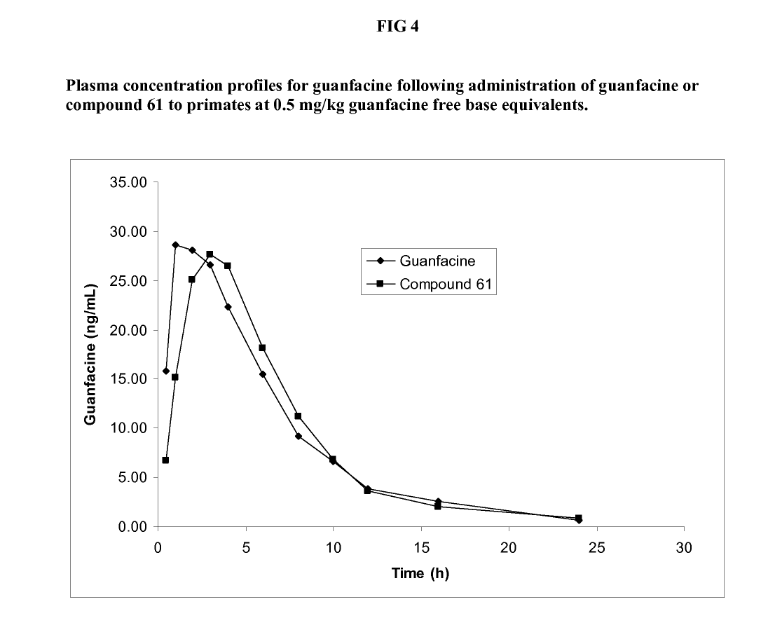

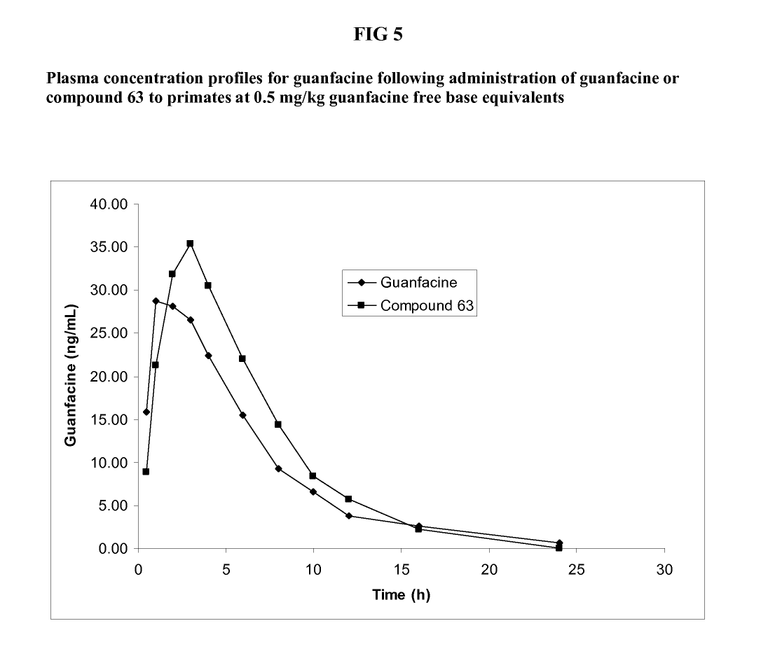

Preferably The present invention is further illustrated by reference to the following Examples. However, it should be noted that these Examples, like the embodiments described above, are illustrative and are not to be construed as restricting the enabled scope of the invention in any way. The bold-faced numbers recited in the Examples correspond to those shown in FIGS. 1-5. Abbreviations are used throughout the examples such as, DCC (dicyclohexylcarbodiimide), NMM (N-methylmorpholine), DME (1,2-dimethoxyethane), NHS (N-hydroxysuccinimide), TFA (trifluoroacetic acid), DSC (N,N′-disuccinimidyl carbonate) and DMF (N,N′-dimethylformamide).

### Example 1

### Preparation of Guanfacine-Glutaryl-Valine Amide (Compound 1)

The synthesis of guanfacine-[glutaryl-(S)-valine] amide trifluoroacetate was accomplished in four steps. Glutaryl-(S)-valine tert-butyl ester was obtained through the reaction of (S)-valine tent-butyl ester with glutaric anhydride. An ‘activated ester’ was prepared from glutaryl-(S)-valine tert-butyl ester by DCC coupling with N-hydroxysuccinimide. The ester was then reacted with guanfacine to give guanfacine-[glutaryl-(S)-valine] amide tert-butyl ester. Removal of the tert-butyl group was achieved by treatment with trifluoroacetic acid to give guanfacine-[glutaryl-(S)-valine] amide trifluoroacetate. The synthetic route is shown in Scheme 1 below.

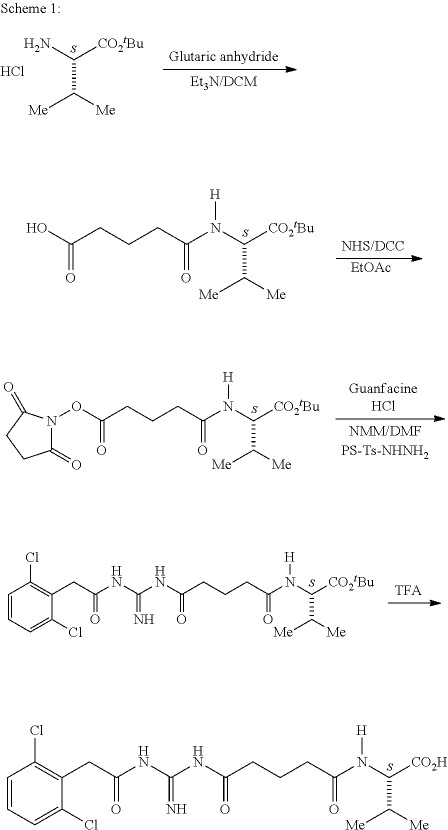

LCMS: m/z=457.00 Consistent for deprotonated ion (M−H)⁻
¹H NMR (DMSO-d₆): 9.72 (br s, 3H, 3×NH), 8.00 (d, J=8.5 Hz, 1H, NH), 7.51 (d, J=7.8 Hz, 2H, 2×ArH), 7.35 (t, J=8.0 Hz, 1H, ArH), 4.15 (m, 1H, α-CH), 4.08 (s, 2H, ArCH₂), 2.45 (m, 2H, CH₂), 2.22 (m, 2H, CH₂), 2.02 (m, 1H, β-CH), 1.77 (m, 2 H, CH₂), 0.89 (m, 6 H, 2×CH₃).

### Example 2

### Synthesis of Guanfacine-β-Alanine-Valine Amide (Compound 2)

The synthesis of guanfacine-β-alanine-(S)-valine amide di-trifluoroacetate was accomplished in six steps. N-Boc-(S)-valine was treated with DCC and N-hydroxysuccinimide to give a first ‘activated ester’ which was then coupled with β-alanine benzyl ester. Subsequent debenzylation afforded N-Boc-(S)-valine-β-alanine and this was then converted to a second ‘activated ester’ by DCC coupling with N-hydroxysuccinimide. This activated ester was coupled with guanfacine to give N-Boc-(S)-valine-β-alanine-guanfacine. Removal of the Boc protecting group was achieved by treatment with trifluoroacetic acid to give guanfacine-β-alanine-(S)-valine amide di-trifluoroacetate. The synthetic route is shown below in Scheme 2.

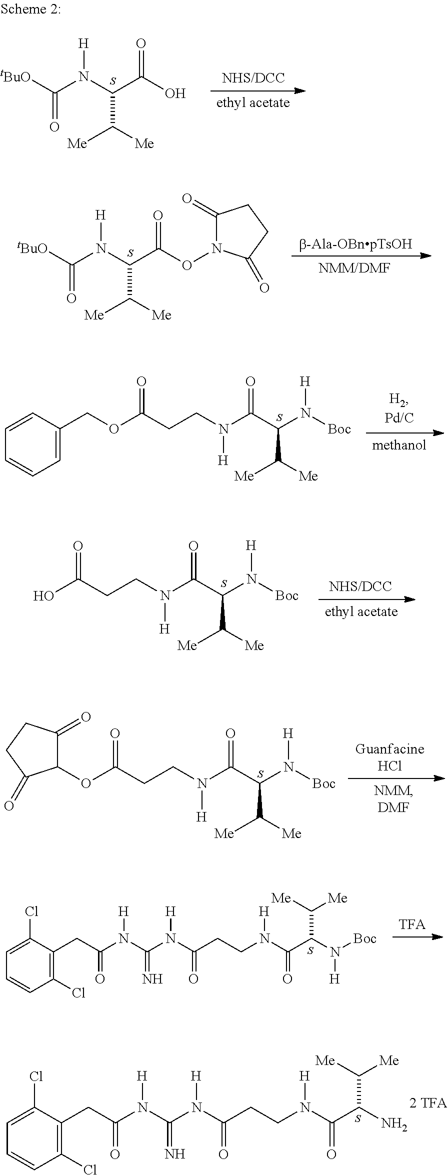

LCMS: m/z=414.00, consistent for deprotonated ion (M−H)⁻
¹H NMR (DMSO-d₆): 9.67 (br, 2H, NH₂), 8.52 (m, 1H, NH), 8.10 (br, 3H, NH₃⁺), 7.51 (d, J=8.0 Hz, 2H, 2×ArH), 7.37 (m, 1H, ArH), 4.07 (s, 2H, ArCH₂), 3.51 (m, 2H, CH₂), 3.33 (m, 1H, α-CH), 2.65 (t, J=6.4 Hz, 2H, CH₂), 2.01 (s, 1H, β-CH), 0.92 (d, J=6.8 Hz, 6H, 2×CH₃).

### Example 3

### Preparation of Guanfacine-γ-Glutamyl-(R)-Valine Amide (Compound 5)

The synthesis of guanfacine-γ-(S)-glutamic acid-(R)-valine amide di-trifluoroacetate was accomplished by a procedure involving six reaction steps. N-Boc-(R)-valine was first treated with DCC and N-hydroxysuccinimide to give a first ‘activated ester’. This ‘activated ester’ was then coupled with H-Glu(OBn)-OtBu and subsequent debenzylation afforded N-Boc-(R)-valine-(S)-glutamic acid tert-butyl ester.

This was converted to a second ‘activated ester’ by DCC coupling with N-hydroxysuccinimide and the ester was reacted with guanfacine to give N-Boc-(R)-valine-(S)-glutamic acid (guanfacine) tert-butyl ester. Removal of the tert-butyl ester and Boc groups was successfully achieved using trifluoroacetic acid to give guanfacine-γ-(S)-glutamic acid-(R)-valine amide di-trifluoroacetate. The synthetic route is shown in Scheme 3.

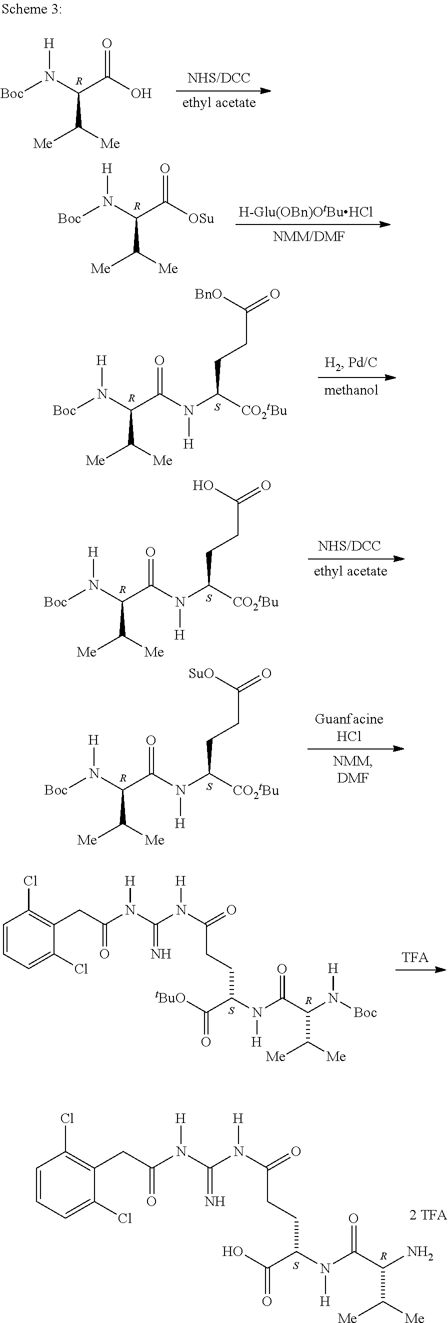

LCMS: m/z=473.96, consistent for protonated ion (MH)⁺
NMR (DMSO-d₆): 9.53 (br, 2H, NH₂⁺), 8.79 (d, J=7.9 Hz, 1H, NH), 8.10 (br, 3H, NH₃⁺), 7.50 (d, J=7.8 Hz, 2H, 2×ArH), 7.34 (m, 1H, ArH), 4.32 (m, 1H, α-CH), 4.05 (s, 2H, ArCH₂), 3.68 (br, 1H, α-CH), 2.50 (2H, obscured, CH₂), 2.11 (m, 2H, CH₂), 1.86 (m, 1H, β-CH), 0.97 (m, 6H, 2×CH₃).

### Example 4

### Preparation of (S)-Serine(Guanfacine)-Sarcosine Carbamate (Compound 61)

The synthesis of (S)-serine(guanfacine)-sarcosine carbamate trifluoroacetate was achieved in six distinct steps. Initially, O-benzyl-(S)-serine was selectively protected by treatment with isobutylene to give (S)-serine(Bn) tert-butyl ester. The protected serine was then coupled to N-Boc-sarcosine N-hydroxysuccinimide ester to yield N-Boc-sarcosine-(S)-serine(Bn) tert-butyl ester. The benzyl ester of serine was deprotected by palladium catalysed hydrogenation followed by activation with N,N′-disuccinimidyl carbonate (DSC) to give an ‘activated carbonate’. The ‘activated carbonate’ was coupled with guanfacine to give N-Boc-sarcosine-(S)-serine(CO.guanfacine) tert-butyl ester. Removal of the Boc and tert-butyl groups was achieved using trifluoroacetic acid to give (S)-serine(guanfacine)-sarcosine carbamate di-trifluoroacetate as a white solid. The synthetic route is shown below in Scheme 4.

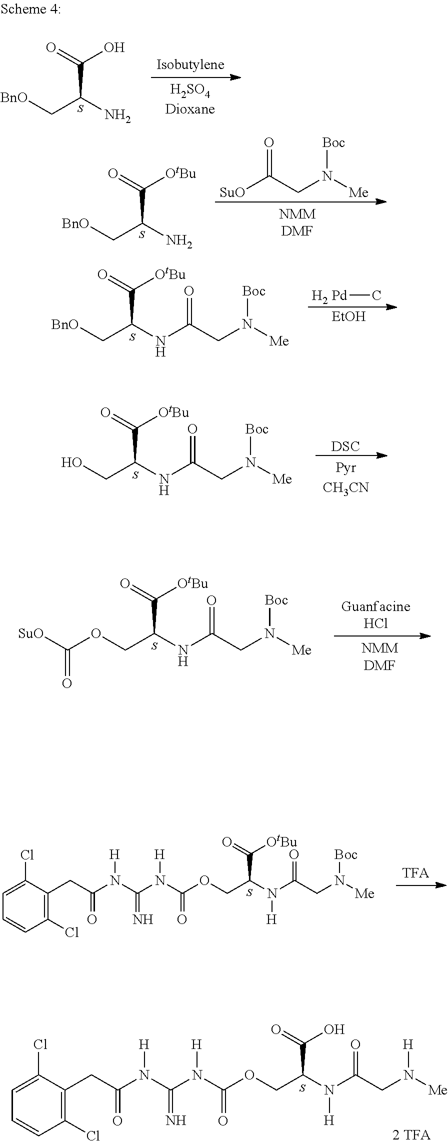

LCMS: m/z=447.85 Consistent for protonated ion (MH⁺)
¹H NMR (DMSO-d₆): 8.95 (d, J=7.8 Hz, 1H, NH), 8.89-8.75 (m, 4H, Guanidine NH₂⁺ and Sarcosine NH₂⁺), 7.50 (d, J=7.8 Hz, 2H, 2×ArH), 7.36 (m, 1H, ArH), 4.62 (m, 1H, Serine α-CH), 4.31 (m, 1H, ½ Serine β-CH₂), 4.21 (m, 1H, ½ Serine β-CH₂), 4.08 (m, 2H, ArCH₂), 3.78 (m, 2H, Sarcosine CH₂), 2.57 (m, 3H, Sarcosine CH₃).

### Example 5

### Preparation of Sarcosine-(2S,3R)-Threonine(Guanfacine) Carbamate (Compound 63)

The synthesis of sarcosine-(2S,3R)-threonine(guanfacine) carbamate di-trifluoroacetate was achieved in six distinct steps. Initially, H-(2S,3R)-threonine(Bn)-OH was selectively protected by treatment with isobutylene to give (2S,3R)-threonine(Bn) tert-butyl ester. The protected threonine was coupled to N-Boc-sarcosine N-hydroxysuccinimide ester to yield N-Boc-sarcosine-(2S,3R)-threonine(Bn) tert-butyl ester. The benzyl ester of threonine was deprotected by palladium catalysed hydrogenation followed by activation with N,N′-disuccinimidyl carbonate (DSC) to give an ‘activated carbonate’. The ‘activated carbonate’ was coupled to guanfacine to give N-Boc-sarcosine-(2S,3R)-threonine(CO.guanfacine) tent-butyl ester. Removal of the Boc and tert-butyl groups was achieved using trifluoroacetic acid to give sarcosine-(2S,3R)-threonine(guanfacine) carbamate di-trifluoroacetate as a white solid. The synthetic route is shown below in Scheme 5.

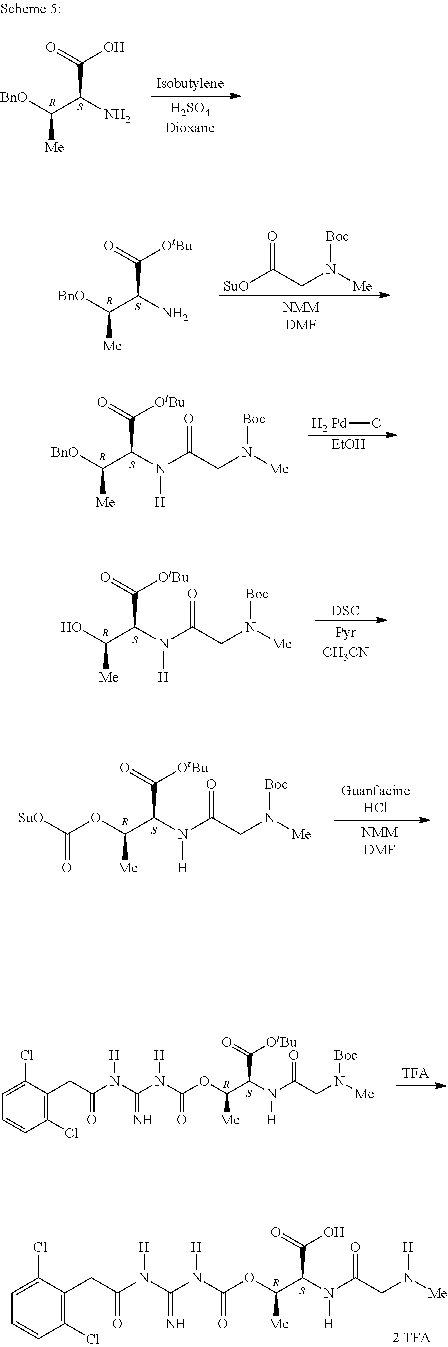

LCMS: m/z=447.90 Consistent for protonated ion (MH⁺)
NMR (DMSO-d₆): 8.83 (m, 2H, NH₂⁺), 8.57 (d, J=7.8 Hz, 1H, NH), 7.72 (br m, 3H, NH₃⁺), 7.51 (d, J=8.4 Hz, 2H, 2×ArH), 7.36 (m, 1H, ArH), 4.55 (m, 1H, Serine α-CH), 4.28 (m, 1H, ½ Serine β-CH₂), 4.17 (m, 1H, ½ Serine β-CH₂), 4.08 (s, 2H, ArCH₂), 2.98 (m, 2H, β-Alanine CH₂), 2.54 (m, 2H, (3-Alanine CH₂).

### Example 6

### Stability of Guanfacine Prodrugs under Conditions Prevailing in the Gastrointestinal (GI) Tract

A high stability of the guanfacine prodrugs in the stomach and intestine is important to avoid local α-2 adrenoceptor agonist effects of the active moiety on the intestinal smooth muscle. A direct action on these receptors in the intestine could be partially responsible for the constipation associated with guanfacine use. If the prodrug were to be prematurely hydrolyzed, the gut would be exposed to the actions of the parent active drug which could lead to a reduction in gut motility. Premature hydrolysis of the guanfacine prodrug would also negate the opportunity to deliver systemically the prodrug from which the active drug might be continuously generated.

Methodology

The rate and extent of hydrolysis of various guanfacine prodrugs was investigated under the conditions prevailing in the GI tract.

Various guanfacine amino acid prodrugs were incubated at 37° C. in simulated gastric and simulated intestinal juice (USP defined composition) for 1 hour and 2 hours, respectively. The remaining concentrations of the prodrugs were then assayed by HPLC.

Results

TABLE 9
Prodrug Stability in simulated gastric and intestinal fluids

| | | % prodrug remaining | |
|---|---|---|---|
| | | following incubation for: | |
| | | 1 h in | 2 h in |
| | | simulated | simulated |
| Pro | Trivial name | gastric fluid | intestinal |
| drug | (Chemical name) | pH 1.1 | fluid pH 6.8 |
| 1 | 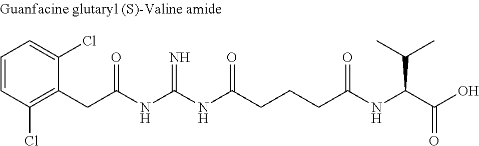 | 80 | 92 |
| 2 | 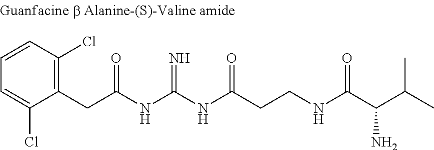 | 75 | 89 |
| 3 | 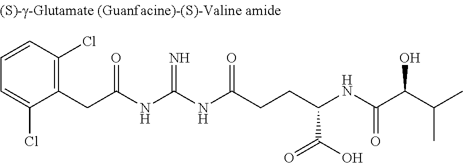 | 87* | 85 |
| 4 | 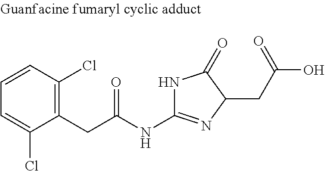 | 97 | 84 |
| 5 | 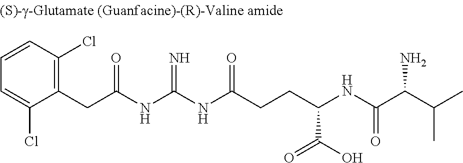 | 86 | 89 |
| 6 | 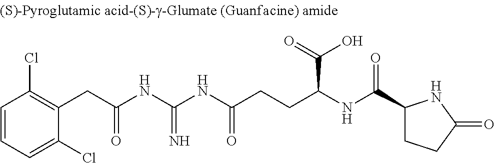 | 81 | 98 |
| 7 | 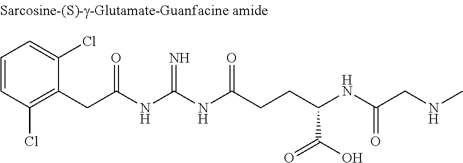 | 79 | 66 |
| 8 | 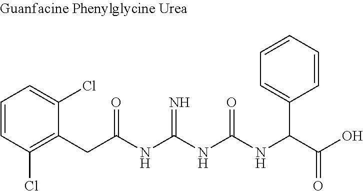 | 96 | 100 |
| 9 | 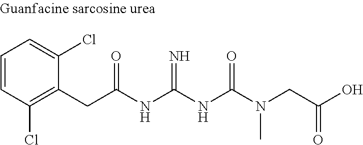 | 101 | 105 |
| 10 | 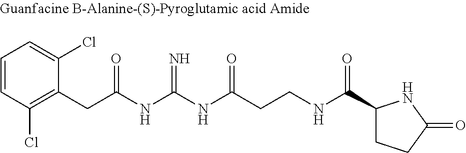 | 82 | 112 |
| 11 | 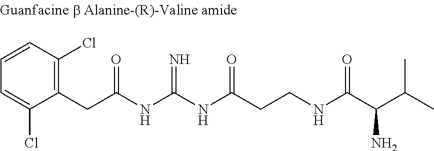 | 79 | 104 |
| 12 | 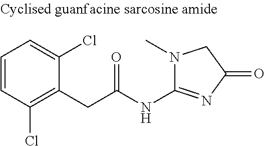 | 48 | 87 |
| 13 | 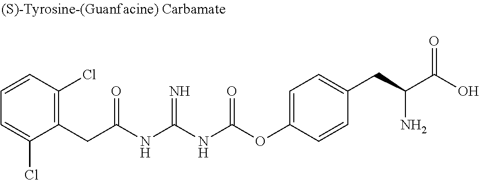 | 74 | 81 |
| 14 | 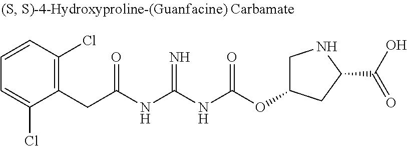 | 70 | 108 |
| 15 | 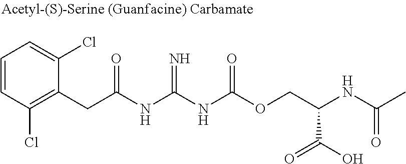 | 83 | 112 |
| 16 | 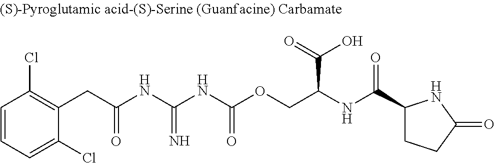 | 78 | 95 |
| 17 | 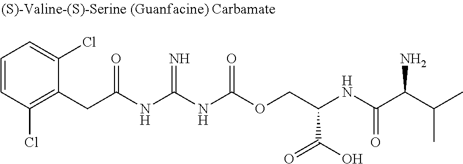 | 93 | 78 |
| 18 | 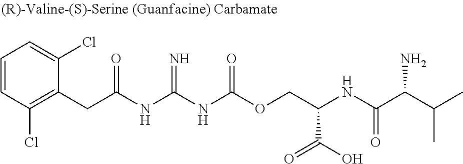 | 74 | 108 |
| 19 | 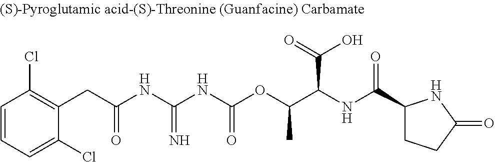 | 92 | 102 |
| 20 | 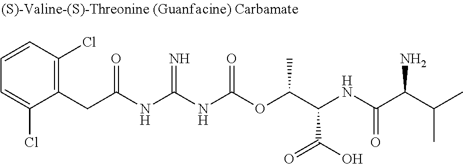 | 79 | 100 |
| 21 | 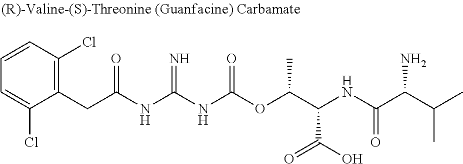 | 83 | 103 |
| 22 | 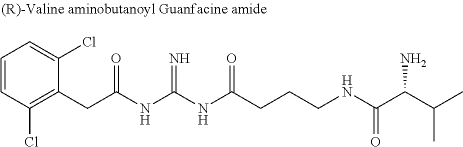 | 106 | 85 |
| 23 | 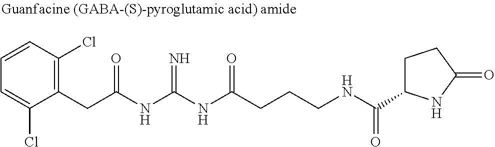 | 98 | 78 |
| 24 | 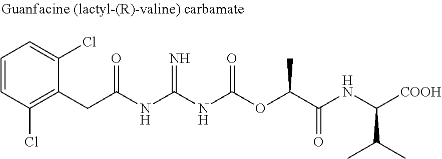 | 104 | 97 |
| 25 | 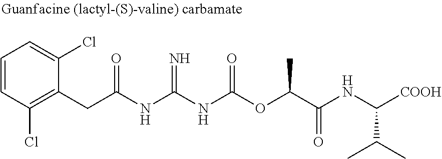 | 101 | 86 |
| 26 | 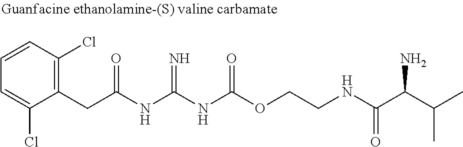 | 97 | 91 |
| 27 | 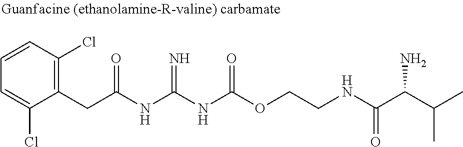 | 78 | 111 |
| 28 | 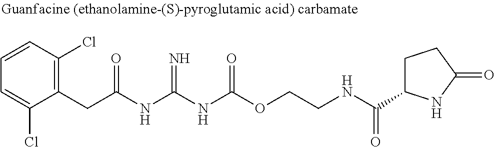 | 103 | 106 |
| 29 | 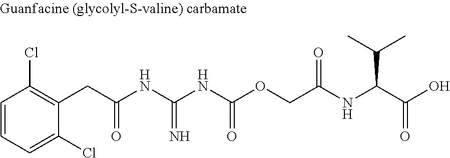 | 80 | 99 |
| 30 | 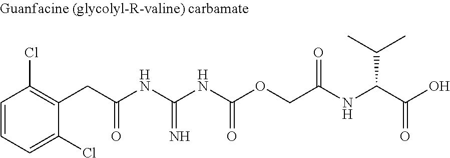 | 77 | 160 |
| 31 | 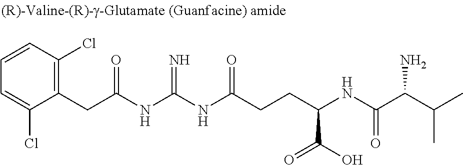 | 70 | 85 |
| 32 | 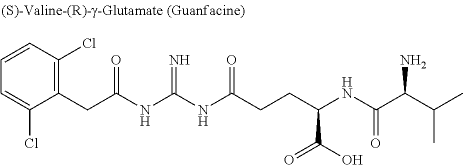 | 93 | 84 |
| 33 | 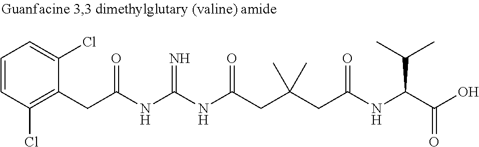 | 79 | 99 |
| 34 | 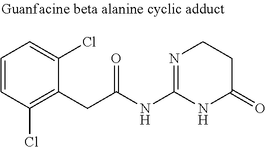 | 51 | 91 |
| 35 | 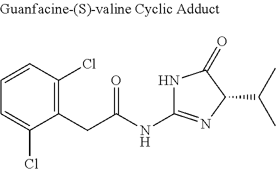 | 83 | 108 |
| 36 | 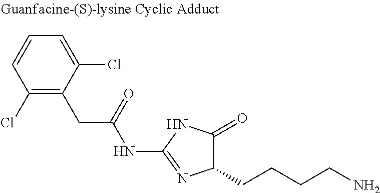 | 83 | 101 |
| 37 | 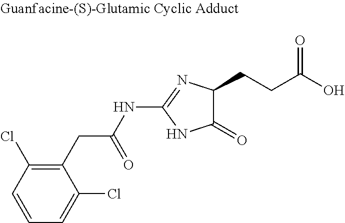 | 58 | 110 |
| 38 | 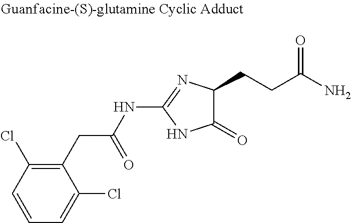 | 70 | 87 |
| 39 | 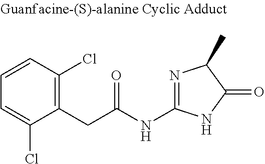 | 89 | 104 |
| 41 | 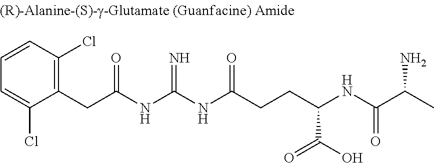 | 70 | 83 |
| 42 | 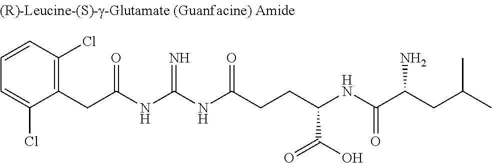 | 70 | 83 |
| 43 | 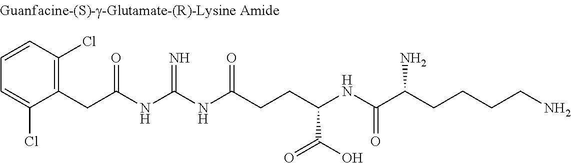 | 62 | 85 |
| 44 | 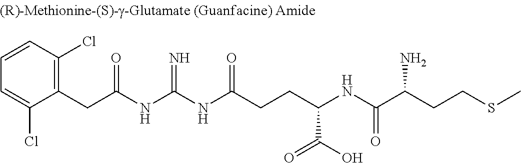 | 75 | 85 |
| 45 | 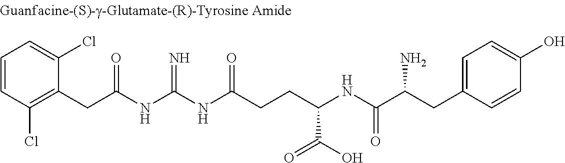 | 74 | 91 |
| 46 | 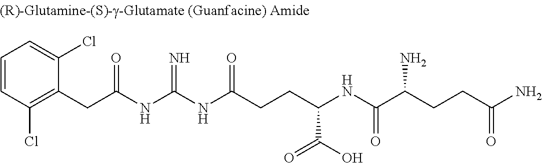 | 61 | 81 |
| 47 | 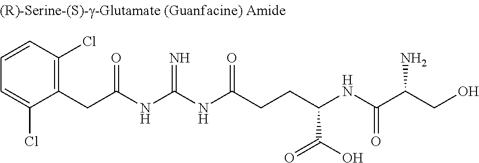 | 72 | 89 |
| 48 | 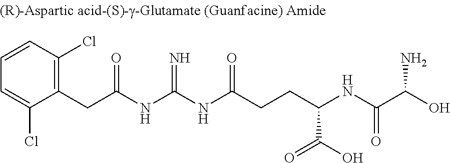 | 80 | 92 |
| 49 | 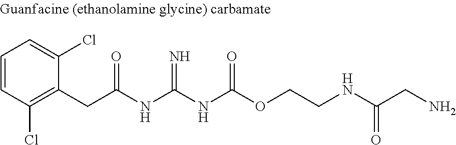 | 87 | 101 |
| 50 | 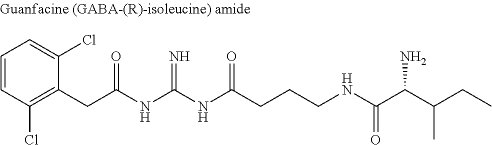 | 85 | 81 |
| 51 | 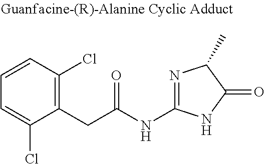 | 80 | 103 |
| 52 | 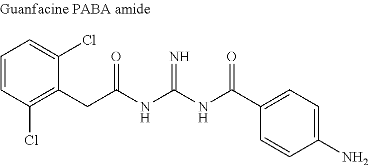 | 84 | 97 |
| 53 | 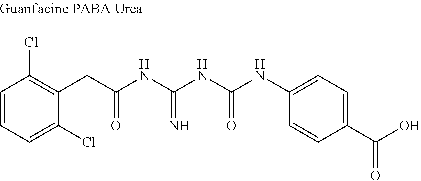 | 101 | 100 |
| 54 | 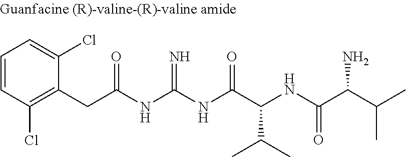 | 73 | 66 |
| 55 | 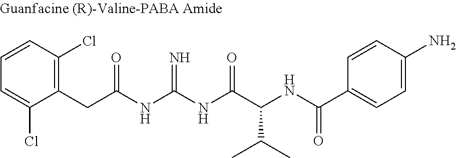 | 65 | 91 |
| 56 | 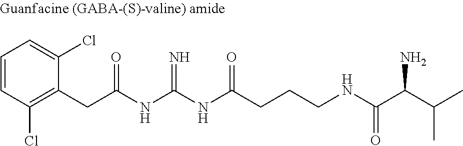 | 70 | 111 |
| 57 | 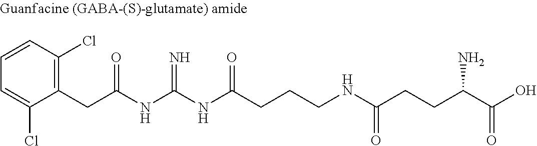 | 81 | 106 |
| 58 | 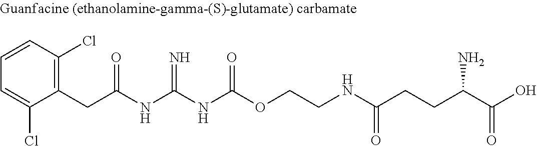 | 96 | 99 |
| 59 | 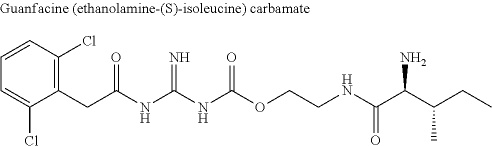 | 92 | 100 |
| 60 | 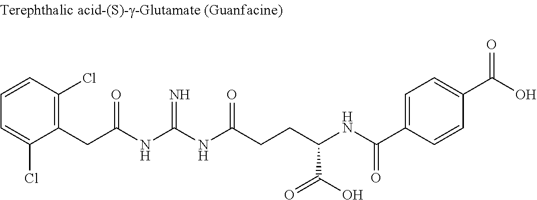 | 81 | 97 |
| 61 | 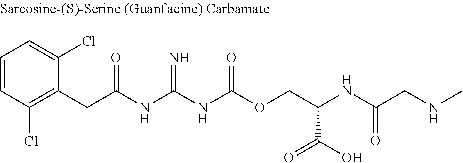 | 92 | 96 |
| 63 | 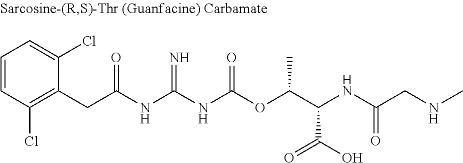 | 86 | 99 |
| 64 | 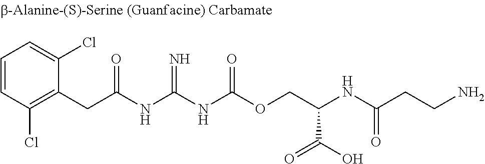 | 88 | 93 |
| 65 | 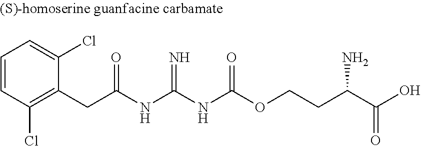 | 90 | 99 |
| 66 | 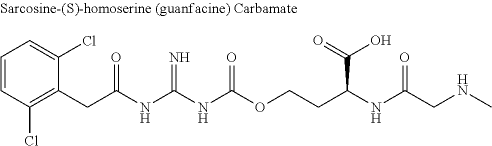 | 86 | 101 |
| 67 | 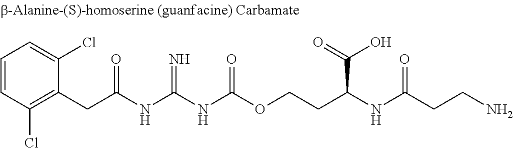 | 99 | 97 |
| 68 | 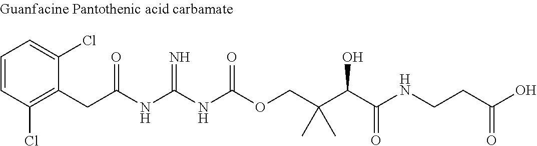 | 86 | 75 |
| 69 | 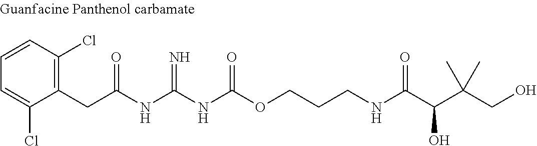 | 85 | 96 |
| 70 | 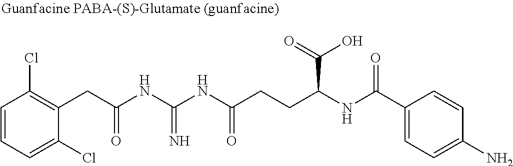 | 64 | 93 |
| 71 | 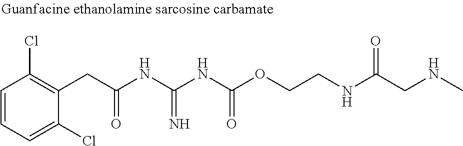 | 91 | 89 |

*2 h incubation period

Many compounds degraded by >40% in either medium and are not shown in Table 9. These include the proteinogenic dipeptide prodrugs of guanfacine conjugated through the alpha carboxylic acid which were commonly quite unstable under the conditions existing in the GI tract although the val-val conjugate did display some limited stability. The N-acetylated amino acid prodrugs of guanfacine conjugated through the alpha carboxylic acid also demonstrated poor stability. The dipeptide prodrugs conjugated to guanfacine through a non-alpha carboxylic acid functional group such as β alanine and γ glutamic acid were more stable. The carbamate-bridged conjugates were generally very stable while the highest stability was observed with urea bridged conjugates and cyclised amino acid derivatives. The dicarboxylic acid bridged amino acid prodrugs and direct amide conjugates displayed intermediate stability.

### Example 7

### Stability of Guanfacine Prodrugs to Enzymes Present in Freshly Withdrawn Porcine Intestinal Fluid

Many of the peptidases in the intestinal lumen may not be present in the USP simulated intestinal fluid preparations previously decribed. Therefore, the rate and extent of hydrolysis of various guanfacine prodrugs was further investigated in porcine intestinal fluid. The prodrugs evaluated were selected on the basis of adequate pharmacokinetics (see Example 8).

Methodology

Various guanfacine amino acid prodrugs were incubated at 37° C. in freshly withdrawn porcine intestinal fluid adjusted to pH 6.8 for 3 hours. The remaining concentrations of the prodrugs and guanfacine formed were then assayed by HPLC.

Results

TABLE 10
Prodrug Stability in Porcine intestinal fluid

| | | Prodrug | Guanfacine |
|---|---|---|---|
| Com- | | remaining | appearing |
| pound | Trivial name | at 3 h | at 3 h |
| 1 | Guanfacine glutaryl (S)-Valine | 100.6 | NM |
| | amide | | |
| 2 | Guanfacine β Alanine-(S)- | 77.8 | NM |
| | Valine amide | | |
| 3 | (S)-Glutamate (Guanfacine)-(S)- | 2.6 | NM |
| | Valine amide | | |
| 5 | (S)-Glutamate(Guanfacine)-(R)- | 91.2 | NM |
| | Valine Amide | | |
| 7 | Sarcosine-(S)-Glutamate- | 48.9 ± 0.8 | 50.6 ± 3.7 |
| | Guanfacine amide | | |
| 11 | Guanfacine β Alanine-(R)- | 91.3 ± 2.6 | 8.4 ± 0.7 |
| | Valine amide | | |
| 17 | (S)-Valine-(S)- | — | 72.2 ± 7.4 |
| | Serine(Guanfacine) Carbamate | | |
| 26 | Guanfacine ethanolamine- | 13.8 ± 0.6 | 100.8 ± 3.1 |
| | (S) valine carbamate | | |
| 41 | (R)-Alanine-(S)- | 89.70 ± 3.60 | 3.90 ± 0.29 |
| | Glutamate(Guanfacine) | | |
| 52 | Guanfacine PABA amide | 87.77 ± 5.80 | — |
| 58 | Guanfacine (ethanolamine-gamma- | 81.26 ± 4.57 | 17.54 ± 0.66 |
| | (S)-glutamate) carbamate | | |
| 61 | Sarcosine-(S)-Serine(Guanfacine) | 66.86 ± 2.10 | 20.12 ± 0.80 |
| | Carbamate | | |
| 63 | Sarcosine-(R,S)-Threonine | 95.04 ± 9.25 | 8.16 ± 0.74 |
| | (Guanfacine) Carbamate | | |

NM = Not measured

Some compounds which showed stability in simulated intestinal fluid were very unstable in porcine intestinal fluid notably 3, 17 and 26. Compounds 2 and 61 showed intermediate stability while compounds 1, 11, 41, 52 and 63 showed a high stability in this medium.

### Example 8

### Comparative Pharmacokinetic Screening Study of Selected Guanfacine Prodrugs in the Monkey

Guanfacine prodrugs with >60% stability in simulated gastric and intestinal fluids were evaluated for conversion to active in cynomolgus monkeys. The monkey showed an absolute oral bioavailability of guanfacine after giving the parent drug of 35%. Although this is lower than the bioavailability of guanfacine in man (>80%), this was higher than in other species tested and the monkey was therefore regarded as the best model for evaluating the pharmacokinetic profiles of the prodrugs.

Test substances e.g. guanfacine (0.5 mg/kg free base) and various guanfacine prodrugs at equimolar doses to that given of the parent drug were administered by oral gavage to groups of two monkeys using a multi-way crossover design.

TABLE 11
Characteristics of experimental animals used in study

| | |
|---|---|
| Species: | Cynomolgus monkey |
| Number and sex: | 2 males per compound |

Blood samples were taken on 4 sampling occasions at various times up to 6 hours after administration and submitted to analysis for the parent drug and prodrug using a qualified LC-MS-MS assay. The relative Cₘₐₓ for guanfacine was calculated by comparison with guanfacine-dosed animals. The results are given in Table 12 below.

TABLE 12
Relative Cₘₐₓ of guanfacine in cynomolgus monkeys
following administration of prodrugs

| | | Relative |
|---|---|---|
| Prodrug | Trivial name | Cmax (%) |
| 1 | Guanfacine glutaryl (S)-Valine Amide | * |
| 2 | Guanfacine β-Alanine-(S)-Valine Am de | * |
| 3 | (S)-γ-Glutamate (Guanfacine)-(S)-Valine Amide | * |
| 4 | Guanfacine fumaryl Cyclic adduct | 0.0 |
| 5 | (S)-γ-Glutamate (Guanfacine)-(R)-Valine Amide | 38.3 |
| 6 | (S)-Pyroglutamic acid-(S)-γ-Glutamate | 23.8 |
| | (Guanfacine) Amide | |
| 7 | Sarcosine-(S)-γ-Glutamate-Guanfacine Amide | 137.8 |
| 8 | Guanfacine Phenylglycine Urea | 0.0 |
| 9 | Guanfacine Sarcosine Urea | 0.0 |
| 10 | Guanfacine B-Alanine-(S)-Pyroglutamic acid | 13.3 |
| | Amide | |
| 11 | Guanfacine β-Alanine-(R)-Valine Amide | 100.0 |
| 12 | Cyclised Guanfacine Sarcosine Amide | 0.0 |
| 13 | (S)-Tyrosine-(Guanfa ine) Carbamate | 26.6 |
| 14 | (S)-4-Hydroxyproline-(Guanfacine) Carbamate | 1.4 |
| 15 | Acetyl-(S)-Serine (Guanfacine) Carbamate | 12.1 |
| 16 | (S)-Pyroglutamic acid-(S)-Serine(Guanfacine) | 14.9 |
| | Carbamate | |
| 17 | (S)-Valine-(S)-Serine(Guanfacine) Carbamate | 113.7 |
| 18 | (R)-Valine-(S)-Serine(Guanfacine) Carbamate | 0.0 |
| 19 | (S)-Pyroglutamic acid-(S)-Threonine(Guanfacine) | 46.9 |
| | Carbamate | |
| 20 | (S)-Valine-(S)-Threonine(Guanfacine) Carbamate | 69.6 |
| 21 | (R)-Valine-(S)-Threonine(Guanfacine) | 0.0 |
| | Carbamate | |
| 22 | (R)-Valine aminobutanoyl Guanfacine Amide | 41.0 |
| 23 | Guanfacine (GABA-(S)-Pyroglutamic acid) Amide | 27.3 |
| 24 | Guanfacine (lactyl-(R)-Valine) Carbamate | 0.0 |
| 25 | Guanfacine (lactyl-(S)-Valine) Carbamate | 23.8 |
| 26 | Guanfacine ethanolamine-(S)-Valine Carbamate | 59.7 |
| 27 | Guanfacine (ethanolamine-(R)-Valine) | 35.4 |
| | Carbamate | |
| 28 | Guanfacine (ethanolamine-(S)-Pyroglutamic acid) | 1.7 |
| | Carbamate | |
| 29 | Guanfacine (glycolyl-(S)-Valine) Carbamate | 1.7 |
| 30 | Guanfacine (glycolyl-(R)-Valine) Carbamate | 0.0 |
| 31 | (R)-Valine-(R)-γ-Glutamate(Guanfacine) Amide | 45.0 |
| 32 | (S)-Valine-(R)-γ-Glutamate (Guanfacine) Amide | 94.9 |
| 33 | Guanfacine 3,3 dimethylglutaryl (Valine) Amide | 0.0 |
| 34 | Guanfacine β Alanine Cyclic adduct | 21.6 |
| 35 | Guanfacine-(S)-Valine Cyclic adduct | 0.0 |
| 36 | Guanfacine-(S)-Lysine Cyclic adduct | 11.2 |
| 37 | Guanfacine-(S)-Glutamic Cyclic adduct | 0.0 |
| 38 | Guanfacine-(S)-Glutamine Cyclic adduct | 9.5 |
| 39 | Guanfacine-(S)-Alanine Cyclic adduct | 22.8 |
| 41 | (R)-Alanine-(S)-γ-Glutamate(Guanfacine) Amide | 86.8 |
| 42 | (R)-Leucine-(S)-γ-Glutamate(Guanfacine) Amide | 59.9 |
| 43 | Guanfacine-(S)-γ-Glutamate-(R)-Lysine Amide | 25.8 |
| 44 | (R)-Methionine-(S)-γ-Glutamate(Guanfacine) | 69.4 |
| | Amide | |
| 45 | Guanfacine-(S)-γ-Glutamate-(R)-Tyrosine Amide | 15.5 |
| 46 | (R)-Glutamine-(S)-γ-Glutamate(Guanfacine) | 51.2 |
| | Amide | |
| 47 | (R)-Serine-(S)-γ-Glutamate(Guanfacine) Amide | 66.5 |
| 48 | (R)-Aspartic acid-(S)-γ-Glutamate(Guanfacine) | 29.0 |
| | Amide | |
| 49 | Guanfacine (ethanolamine Glycine) Carbamate | 95.8 |
| 50 | Guanfacine (GABA-(R)-Isoleucine) Amide | 40.3 |
| 51 | Guanfacine-(R)-Alanine Cyclic adduct | 52.2 |
| 52 | Guanfacine PABA Amide | 56.6 |
| 53 | Guanfacine PABA Urea | 0.0 |
| 54 | Guanfacine (R)-Valine-(R)-Valine Amide | 31.4 |
| 55 | Guanfacine (R)-Valine-PABA Amide | 0.0 |
| 56 | Guanfacine (GABA-(S)-Valine) Amide | 63.5 |
| 57 | Guanfacine (GABA-(S)-Glutamate) Amide | 59.6 |
| 58 | Guanfacine (ethanolamine-(S)-γ-Glutamate) | 57.4 |
| | Carbamate | |
| 59 | Guanfacine (ethanolamine-(S)-Isoleucine) | 57.1 |
| | Carbamate | |
| 60 | Terephthalic acid-(S)-γ-Glutamate(Guanfacine) | 21.6 |
| | Amide | |
| 61 | Sarcosine-(S)-Serine(Guanfacine) Carbamate | 93.1 |
| 63 | Sarcosine-(R,S)-Threonine (Guanfacine) | 80.4 |
| | Carbamate | |
| 64 | β-Alanine-(S)-Serine (Guanfacine) Carbamate | 49.9 |
| 65 | (S)-Homoserine Guanfacine Carbamate | 35.0 |
| 66 | Sarcosine-(S)-Homoserine (Guanfacine) | 35.4 |
| | Carbamate | |
| 67 | β-Alanine-(S)-Homoserine (Guanfacine) | 49.6 |
| | Carbamate | |
| 68 | Guanfacine Pantothenic acid Carbamate | 30.2 |
| 69 | Guanfacine Panthenol Carbamate | 21.0 |
| 70 | Guanfacine PABA-(S)-Glutamate (Guanfacine) | 8.7 |
| | Amide | |
| 71 | Guanfacine ethanolamine-Sarcosine Carbamate | 39.0 |

* Not available; data from full pharmacokinetic studies is presented in Example 9

A relative Cmax>30% was considered a favourable attribute as this indicates that the prodrug will be less prone to a high interindividual variation in circulating levels of the active drug after oral administration.

A high relative guanfacine Cmax but total absence of prodrug in the plasma suggested that the prodrug was converted to guanfacine in the intestinal lumen prior to absorption. This was the case for prodrugs such as compound 17.

A low relative guanfacine Cmax with high prodrug levels suggested adequate stability and absorption of prodrug but poor subsequent conversion to the active. This was the case for prodrugs such as compounds 9 and 37.

A moderate to high relative guanfacine Cmax with detectable prodrug levels in plasma suggested that the prodrug could be absorbed intact and then efficiently converted to guanfacine as exemplified by compounds 2, 11, 41, 61 and 63.

### Example 9

### Comparative Bioavailability Study of Guanfacine in Monkeys Given Either Guanfacine Itself or Various Guanfacine Prodrug Conjugates

In order to characterize the pharmacokinetics of selected guanfacine conjugates fully, test substances e.g. guanfacine (0.5 mg/kg) and various guanfacine prodrug conjugates were administered by oral gavage at equimolar doses to groups of five cynomolgus monkeys using a multi-way crossover design. The characteristics of the test animals are set out in Table 13.

TABLE 13
Characteristics of experimental monkeys used in study

| | |
|---|---|
| Species: | Monkey |
| Type: | Cynomolgus |
| Number and sex: | 5 males per group |

Blood samples were taken at various times after administration and submitted to analysis for the parent drug and prodrug using a qualified LC-MS-MS assay. The following pharmacokinetic parameters derived from the plasma analytical data were determined using Win Nonlin;

| | |
|---|---|
| Cmax | Maximum measured concentration |
| Tmax | Time at which maximum concentration was apparent |
| T50% > Cmax | Time period which plasma guanfacine concentrations |
| | remain at or above 50% of Cmax |
| Frel % | Relative oral bioavailability of Guanfacine |

The results are given in Table 14 below and FIGS. 1 to 5.

TABLE 14
Guanfacine pharmacokinetic parameters following administration
of guanfacine or prodrugs to the cynomolgus monkeys

| | | | | Prodrug Cmax/ |
|---|---|---|---|---|
| Compound | Fᵣₑₗ % | Tₘₐₓ | T50% > Cmax | Guanfacine Cmax |
| 1 | 29 | 3.4 | 15.1 | 1.24 |
| 2 | 61 | 1.6 | 5.2 | 0.08 |
| 3 | 73 | 2.4 | NM | ND |
| 5 | 60 | 6 | 22 | 0.23 |
| 41 | 101 | 2.4 | 4.5 | 0.13 |
| 61 | 110 | 3 | 4 | 0.16 |
| 63 | 130 | 3.2 | 4.6 | 0.31 |

ND: Prodrug not detected;
NM: Not measured.

In studies investigating the pharmacokinetics of guanfacine under identical conditions, the Tₘ>50% Cmax averaged 4.9 h.

The administration of compounds 2, 61, and 63 resulted in a plasma guanfacine profile similar to that seen after the parent drug with fairly rapid attainment of Tmax and a corresponding mean Cmax greater than 75% of that seen after the parent drug. Similarly a high mean bioavailability over 60%, relative to that observed after giving the parent drug, was observed for compounds 2, 61, 63 and 41. For all compounds except compound 3 prodrug was detected in the plasma demonstrating absorption of the prodrug.

Administration of compounds 1 and 5 resulted in sustained guanfacine concentrations as demonstrated by the prolonged T50%>Cₘₐₓ values relative to guanfacine with consequently lower Cmax values. Such a pharmacokinetic profile with lower Cₘₐₓ values would potentially minimise the possibility of unwanted CNS and cardiovascular effects.

Dosage with compound 3 resulted in a plasma concentration time profile and exposure very similar to that seen after giving guanfacine itself. The mean relative bioavailability was ˜75%, however, the lack of prodrug in the plasma suggested that the conversion to guanfacine may have occurred in the gastrointestinal lumen prior to absorption.

These representative examples of different classes of guanfacine prodrugs demonstrate that only selected amino acid conjugates are capable of delivering substantial amounts of guanfacine into the systemic circulation and fewer still are capable of delivering sustained levels of active drug compared to dosing of oral guanfacine.

### Example 10

### Comparative Bioavailability Study of Guanfacine in Monkeys Given Either Guanfacine Itself or Various Guanfacine Prodrug Conjugates in the Fasted or Fed State

The controlled release form of guanfacine INTUNIV® is considered to be subject to a food interaction. Administration of INTUNIV® with a high fat meal has been shown to elevate Cₘₐₓ by 75% and increase AUC by 40% (FDA label). While taking the drug under more appropriate prandial conditions may be desirable, this may not always be possible. Variations in the prandial state may therefore lead to some variability in rate and extent of drug exposure. Guanfacine prodrugs should therefore ideally be devoid of such a food interaction in order to deliver similar guanfacine levels in the fed and fasted state.

Methodology

Five male cynomolgus monkeys were used. Food was withdrawn from animals in fasted groups from the evening of the day prior to dosing until approximately 4 hours after dosing. The vehicle for the compounds was sterile water for irrigation (guanfacine and compound 2) or 0.5% carboxymethylcellulose (compound 1).

The formulations were prepared on the day of dosing and administered orally as soon as practicable up to a maximum of 2 hours after formulation. Animals were dosed at 0.5 mg/kg guanfacine free base equivalents.

Blood samples (0.5 mL) were collected from all animals pre-dose and at 0.5, 1, 2, 3, 4, 6, 8 10, 12, 16 and 24 hours after dosing.

Following processing, the resultant plasma was frozen and analysed by a qualified method. Pharmacokinetic evaluation was performed using a validated pharmacokinetic software package.

Following oral administration of the prodrugs, food did not affect the rate and extent of absorption of the prodrugs and the extent of formation of guanfacine.

TABLE 15
Guanfacine pharmacokinetic parameters following
administration of guanfacine or prodrugs to the
cynomolgus monkey in fasted or fed condition

| | Guanfacine | | | | |
|---|---|---|---|---|---|
| | equivalent | | mean Cₘₐₓ (ng/mL) | | |
| Test compound | dose (mg/kg) | Food status | guanfacine | prodrug | Fᵣₑₗ % |
| Guanfacine | 0.5 | Fasted | 31.3 | N/A | N/A |
| Guanfacine | 0.5 | Fed | 30.2 | N/A | N/A |
| 1 | 0.5 | Fasted | 6.82 | 9.23 | 27.0 |
| 1 | 0.5 | Fed | 6.11 | 7.19 | 27.0 |
| 2 | 0.5 | Fasted | 20.5 | BLQ | 59.0 |
| 2 | 0.5 | Fed | 17.7 | 2.15 | 53.0 |

N/A—Not applicable

The apparent absence of a food effect for guanfacine is a consequence of its administration as unformulated guanfacine in place of the commercial sustained release formulation.

### Example 11

### The Pharmacokinetics of Guanfacine and Prodrugs in Rats in Hepatic Portal and Tail Veins Following Oral Administration of Guanfacine or Prodrug

The absorption of intact prodrug and conversion of prodrug to guanfacine after absorption is important if any local effects of the active compound on alpha 2 adrenoceptors in the gastrointestinal tract are to be minimised. The collection of blood from the hepatic portal vein following oral administration allows the analysis of absorbed prodrug and active drug levels prior to first pass metabolism in the liver. Systemic levels can be measured by sampling of blood from the tail vein.

Methodology

Rats were surgically prepared under isofluorane anaesthesia by attaching a silicon catheter to the portal vein then exteriorising it at the nape of the neck with a blood collection port attached.

Oral doses of guanfacine or prodrug were administered by gavage as a single bolus dose at a dose volume of 10 mL/kg.

At each sampling time serial point blood samples (approximately 0.2 mL) were taken simultaneously from the lateral tail vein cannula and the hepatic portal cannula. After collection of the final blood sample each animal was killed by cervical dislocation. Blood samples were collected at 15, 30 minutes and 1, 2, 4, 8 and 24 hours post dose.

Pharmacokinetic parameters in portal and systemic plasma were derived by non-compartmental analysis (linear/logarithmic trapezoidal) using WinNonlin (Version 4.1) software.

Results

TABLE 16
Guanfacine conjugates; Pharmacokinetic parameters in hepatic portal vein and tail vein
following oral administration to rat at 1 mg/kg guanfacine free base equivalents

| | Hepatic portal vein | | | | Tail vein | | | |
|---|---|---|---|---|---|---|---|---|
| | Prodrug | | Guanfacine | | Prodrug | | Guanfacine | |
| | Cmax | AUC | Cmax | AUC | Cmax | AUC | Cmax | AUC |
| Compound | (ng/mL) | (ng · h/mL) | (ng/mL) | (ng · h/mL) | (ng/mL) | (ng · h/mL) | (ng/mL) | (ng · h/mL) |
| Guanfacine | | | 304 | 290 | | | 18.9 | 29.8 |
| Guanfacine* | | | 114 | 107.1 | | | 3.94 | 10.5 |
| 1* | 56.3 | 159.1 | 13.8 | 42.9 | 8.26 | 29.6 | 0.704 | 3.4 |
| 2 | 46.4 | 19.25 | 35.8 | 41.3 | 4.2 | 2.67 | 5.1 | 13.1 |
| 5 | 41.5 | 61 | 43.1 | 93.9 | 3.5 | 4.6 | 1.4 | 1 |
| 41 | 31.9 | 43.5 | 88.6 | 232 | 6.14 | 8 | 7.31 | 25.2 |
| 61 | 43.4 | 51.8 | 55.3 | 237 | 7.07 | 5.09 | 4.39 | 31.2 |
| 63 | 23.7 | 66.5 | 89.9 | 330 | 9.37 | 20.2 | 6.26 | 39.1 |

*0.5 mg/kg

The substantial presence of the prodrug in the hepatic portal circulation relative to the concentration in the systemic circulation demonstrated the absorption of the prodrug prior to absorption across the intestine and confirmed adequate stability in the intestinal lumen. This suggests a lack of extensive degradation of the prodrugs prior to absorption and a reduction in the potential to elicit a direct pharmacological effect in the gut lumen.

### Example 12

### In Vitro Assessment of the Effects of Guanfacine and Selected Prodrugs on α-2A Adrenoceptor Binding

The target receptor for guanfacine is the human α-2A adrenoceptor subtype in the central nervous system. The activation of this receptor is responsible for its intended therapeutic effect. However, it is possible that local activation of α-2A adrenoceptors present in the gut contributes to adverse gastrointestinal effects (constipation) associated with guanfacine. The receptor binding of the prodrugs was investigated to confirm that the prodrug molecules had been largely inactivated.

Methods

The binding assay methodology employed in this study followed that described by Langin et al.

(Eur. J. Pharmacol. 167:95-104, 1989) and used human recombinant CHO cells expressing α-2 adrenoceptors. The competitive binding ligand was [3H] RX821002 (1 nM) which has a high affinity for the alpha-2A subtype.

Results

The results are set forth in Table 17. Guanfacine in non-prodrug form showed considerable potency as a competitive binding agent at the α-2A adrenoceptor displaying an Ki of 32 nM. The prodrugs tested in the assay were all less potent binding agents to the receptor with most displaying Ki values greater than 30-fold those obtained with guanfacine. Thus, the prodrugs described herein would have little or no effect on intestinal α-2A adrenoceptors and hence potentially have a diminished ability to induce constipation through direct actions on gut motility, compared to guanfacine in non-prodrug form.

TABLE 17
Binding of guanfacine and various prodrugs at α-2A adrenoceptor

| Compound | Ki (nM) |
|---|---|
| Guanfacine | 32 |
| 2 | 1200 |
| 1 | 7200 |
| 41 | 1700 |
| 5 | 1600 |
| 61 | 10000 |

### Example 13

### In vivo Effects of Guanfacine and its Prodrugs on Gut Motility in Rat

The effect of a drug on gut motility can be studied by means of the charcoal propulsion test. Drugs known to cause constipation such as morphine and guanfacine significantly delay the transit of a charcoal meal in the rat. The effects of guanfacine in non-prodrug form and its prodrugs on GI motility were assessed in groups of 10 rats fasted overnight prior to the test.

The method used was based on that described by Takemori et al. (J. Pharmacol. Exp. Ther. 169:39, 1969). Test treatments were administered orally 60 minutes prior to an oral dose of a 10% suspension of charcoal in 2.5% gum Arabic (2 ml/kg). Twenty minutes after dosing with charcoal, the rats were sacrificed and the entire gastrointestinal tract was removed quickly and carefully. The distance that the charcoal meal had travelled toward the caecum was measured and expressed as a percentage of the total gut length. The results are descibed in Table 18.

Orally administered guanfacine in non-prodrug form at a dose of 0.1 mg base/kg had significant effects on gut motility with 41-52% reduction in the distance travelled by the charcoal plug within 20 minutes, compared to that of the control group (treated with the vehicle). All the prodrugs were considerably less potent than guanfacine in the inhibition of GIT transit in the rat. Notably the doses of compounds 2, 61 and 63 required to inhibit G IT transit to the same extent as guanfacine were 10-fold or greater expressed as molar equivalents. The comparative systemic exposure to guanfacine in rats following oral administration of compounds 61 and 63 was similar to that following guanfacine administration at an equimolar dose. For compound 2 the systemic guanfacine exposure was ca 40% compared to guanfacine administration.

TABLE 18
Effects of guanfacine or prodrugs on gastrointestinal
transit of a charcoal meal in the rat

| Dose (guanfacine free base equivalents) mg/kg | | | | | | | |
|---|---|---|---|---|---|---|---|
| 0.03 | 0.1 | 0.3 | 0.5 | 1 | 1.7 | 3 | 5 |

| | |
|---|---|
| Compound | % inhibition of GIT transit compard to vehicle |

Study 1

| | | | | | | |
|---|---|---|---|---|---|---|
| Guanfacine | −52 | −55 | −47 | | | |
| 2 | +3 | −30 | −42 | | −42 | −42 |
| 1 | −9 | −6 | −45 | | −50 | −53 |
| 5 | −6 | −22 | −48 | −50 | −44 | −42 |

Study 2

| | | | | |
|---|---|---|---|---|
| Guanfacine | −13 | −41 | −56 | |
| 61 | | −8 | −25 | −31 |
| 63 | | 0 | −13 | −49 |

Without being by bound by any theory, the lack of effects on gut motility by the prodrugs is attributed in part to the reduced or minimally availabile active drug (guanfacine) within the gut lumen to interact locally with α-2 adrenoceptors.

1. A guanfacine prodrug of Formula (I), or a pharmaceutically acceptable salt or tautomer thereof: 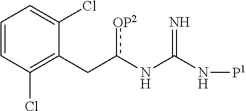
wherein:
P¹ is hydrogen or -L-R;
P² is absent, hydrogen or -L-R;
provided that when P is hydrogen, P² is not absent;
L is absent, or a group selected from the group consisting of 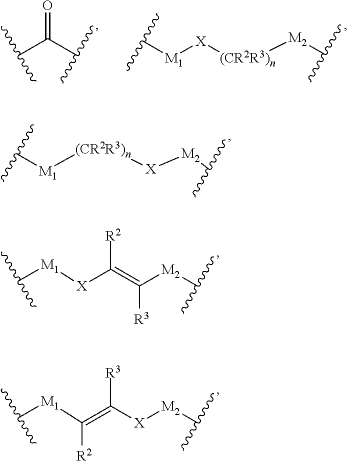
an amino acid residue containing from 2 to 20 carbon atoms, and a peptide formed from 2 to 10 independently selected amino acids each containing from 2 to 20 carbon atoms;
wherein:
M₁ is absent or is selected from the group consisting of —CH₂—, 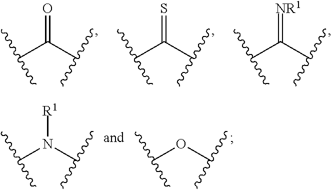
wherein R¹ is selected from the group consisting of H, C₁₋₄ alkyl and C₃₋₈ cycloalkyl;
M₂ is absent or is selected from the group consisting of —CH₂—, 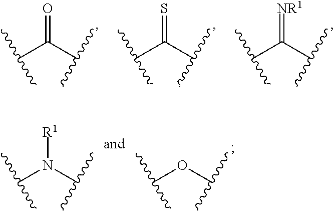
wherein R¹ is selected from the group consisting of H, C₁₋₄ alkyl and C₃₋₈ cycloalkyl;
R² and R³ are each independently selected at each occurrence from the group consisting of hydrogen, hydroxy, C₁₋₆ alkoxy, C₁₋₆ alkyl C₁₋₆ alkoxy, —(CR⁴R⁵)ₙOC(═O)R⁶, —(CR⁴R⁵)ₙC(═O)R⁶, —C(═O)R⁶, C₁₋₆ alkyl, C₁₋₆ haloalkyl, aryl, —NR⁴R⁵ and —NR⁴(CO)R⁶; or together with the atom to which they are bonded, R² and R³ may form a carbonyl, an ethylene or a C₃₋₆ cycloalkyl;
R⁴ and R⁵ are each independently selected from the group consisting of H, C₁₋₆ alkyl, C₁₋₆ haloalkyl, C₃₋₈ cycloalkyl and phenyl;
R⁶ is selected from the group consisting of hydroxyl, C₁₋₆ alkyl, C₁₋₆ alkoxy, C₃₋₈ cycloalkyl and phenyl;
X is selected from the group consisting of a bond, —O—, —NH—, —CR²R³— and a saturated or unsaturated ring having from 3 to 6 carbon atoms in the ring;
R is hydroxy, an amino acid residue containing from 2 to 20 carbon atoms or a peptide formed from 2 to 10 independently selected amino acids each containing from 2 to 20 carbon atoms, or R is a group selected from the group consisting of —NH₂ and —NR⁴R⁵; and
n is at each occurrence independently an integer of 0-16.

2. A guanfacine prodrug of Formula (I), as claimed in claim 1, wherein:
P¹ is hydrogen or -L-R;
P² is absent, hydrogen or -L-R;
provided that when P² is hydrogen, P² is not absent;
L is absent, or a group selected from the group consisting of 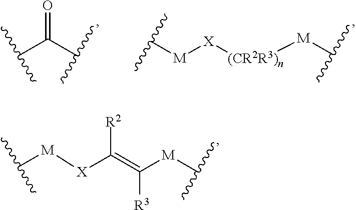
an amino acid residue containing from 2 to 20 carbon atoms, and a peptide formed from 2 to 10 independently selected amino acids each containing from 2 to 20 carbon atoms; wherein:
each M is independently absent or independently selected at each occurrence from the group consisting of —CH₂—, 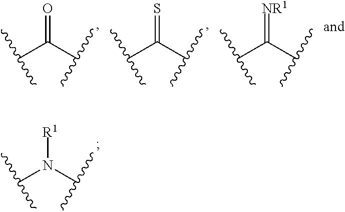
wherein R¹ is selected from the group consisting of H, C₁₋₄ alkyl and C₃₋₈ cycloalkyl;
R² and R³ are each independently selected at each occurrence from the group consisting of hydrogen, hydroxy, C₁₋₆ alkoxy, C₁₋₆ alkyl alkoxy, —(CR⁴R⁵)ₙOC(═O)R⁶, —C(═O)R⁶, C₁₋₆ alkyl, C₁₋₆ haloalkyl, aryl, —NR⁴R⁵ and —NR⁴(CO)R⁶; or together with the atom to which they are bonded, R² and R³ may form a C₃₋₆ cycloalkyl;
R⁴ and R⁵ are each independently selected from the group consisting of H, C₁₋₆ alkyl, C₁₋₆ haloalkyl, C₃₋₈ cycloalkyl and phenyl;
R⁶ is selected from the group consisting of hydroxyl, C₁₋₆ alkyl, C₁₋₆ alkoxy, C₃₋₈ cycloalkyl and phenyl;
X is selected from the group consisting of a bond, —O— and —NH—;
R is hydroxy, an amino acid residue containing from 2 to 20 carbon atoms or a peptide formed from 2 to 10 independently selected amino acids each containing from 2 to 20 carbon atoms, or R is a group selected from the group consisting of —NH₂ and —NR⁴R⁵; and
n is at each occurrence independently an integer of 0-10.

3. A guanfacine prodrug as claimed in claim 1,
wherein
P¹ is -L-R and P² is absent.

4. A guanfacine prodrug as claimed in claim 1, wherein L is selected from the group consisting of

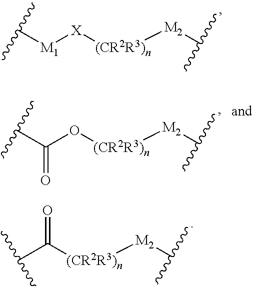

5. (canceled)

6. A guanfacine prodrug as claimed in claim 1, wherein M₂ is

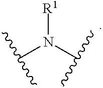

7-9. (canceled)

10. A guanfacine prodrug as claimed in claim 1, wherein R² and R³ are each independently selected at each occurence from the group consisting of H, —OH, C₁₋₆ alkyl and —C(═O)R⁶, or R² and R³ together with the atom to which they are bonded form a carbonyl group.

11. A guanfacine prodrug as claimed in claim 1, wherein, n is independently selected at each occurence from the value 0, 1, 2, 3 or 4.

12. A guanfacine prodrug as claimed in claim 1, wherein R is an amino acid residue containing from 2 to 20 carbon atoms.

13. A guanfacine prodrug as claimed in claim 12, wherein R is selected from the group consisting of valine, N—C₁₋₆ alkylated valine, N,N-C₁₋₆ dialkylated valine, N-methyl valine, N,N-dimethyl valine, alanine, N-C₁₋₆ alkylated alanine, N,N-C₁₋₆ dialkylated alanine, N-methyl alanine, N,N-dimethyl alanine, leucine, N-C₁₋₆ alkylated leucine, N,N-C₁₋₆ dialkylated leucine, N-methyl leucine, N,N-dimethyl leucine, isoleucine, N-C₁₋₆ alkylated isoleucine, N,N-C₁₋₆ dialkylated isoleucine, N-methyl isoleucine, N,N-dimethyl isoleucine, glycine, N-C₁₋₆ alkylated glycine, N,N-C₁₋₆ dialkylated glycine, N-methyl glycine, N-methylcyclopropyl glycine, N,N-dimethyl glycine, and N,N-dimethylcyclopropyl glycine.

14. A guanfacine prodrug as claimed in claim 1, wherein L is 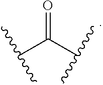
and R is a peptide and is selected from the group consisting of serine-glycine, serine-alanine, serine-dimethyl glycine, serine-dimethylcyclopropyl glycine, serine-sarcosine, threonine-glycine, threonine-alanine, threonine-dimethyl glycine, threonine-dimethylcyclopropyl glycine and threonine-sarcosine.

15. A guanfacine prodrug selected from the group consisting of guanfacine-[glutaryl-(S)-valine] amide 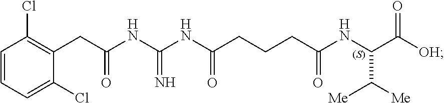
guanfacine-[β-alanine-(S)-valine] amide 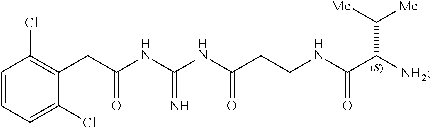
guanfacine-[γ-(S)-glutamic acid-(R)-valine] amide 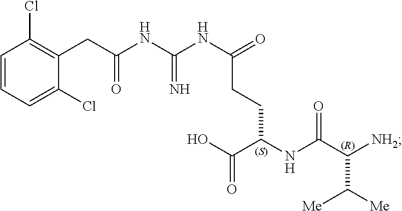
(S)-serine(guanfacine)-sarcosine carbamate 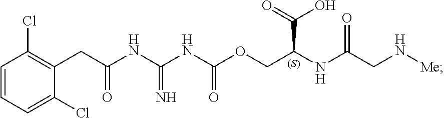
and
sarcosine-(2S,3R)-threonine(guanfacine) carbamate

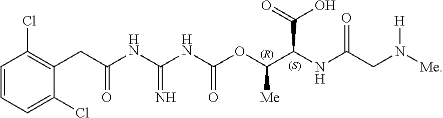

16. A guanfacine prodrug of claim 1 or a pharmaceutically acceptable salt thereof, wherein when ingested orally, the prodrug induces statistically significantly lower average effects on gut motility in the gastrointestinal environment than a non-prodrug guanfacine salt form.

17. A pharmaceutical composition comprising a guanfacine prodrug of claim 1.

18. A method for treating a condition selected from the group consisting of attention deficit hyperactivity disorder (ADHD), oppositional defiance disorder (ODD), a cardiovascular condition such as hypertension, neuropathic pain, cognitive impairment associated with schizophrenia (CIAS), anxiety (including PTSD, OCD, self injury), addiction withdrawal, autism, chemotherapy induced mucositis, post traumatic stress syndrome, and a disorder characterized by the patient suffering from hot flushes, comprising administering an effective amount of a guanfacine prodrug of claim 1 to a subject in need thereof.

19. A method of claim 18 wherein the condition is attention deficit hyperactivity disorder (ADHD).

20-21. (canceled)

22. A method of reducing gastrointestinal side effects associated with guanfacine therapy in a mammal, comprising:
(a) forming a guanfacine prodrug of claim 1 or a pharmaceutically acceptable salt thereof; and
(b) administering the prodrug or a pharmaceutically acceptable salt thereof to a mammal in need thereof.

23-25. (canceled)

26. The method of claim 22, wherein when ingested orally, the prodrug induces statistically significantly lower average effects on gut motility in the gastrointestinal environment than a non-prodrug guanfacine salt form.

27. The method of claim 22, wherein the prodrug or a pharmaceutically acceptable salt thereof is administered orally.

28. The method of claim 22, wherein the prodrug or a pharmaceutically acceptable salt thereof is administered in an amount of from about 1 to about 10 mg based on the amount of guanfacine in free base form.

29. The method of claim 22, wherein the Ki of the prodrug or a pharmaceutically acceptable salt thereof is at least 10 fold greater than the Ki of guanfacine in competitive binding to α-2A adrenoceptors.
